SEQUENCE LISTING (21 sequences)

SEQ ID NO: 1 - protein, 122 aa, synthetic construct
QVQLQESGGGLVQTGGSLRLSCAASGSFFSINDMGWYRQAPGKQRELVAMITGDDSTNYADSVKGRFTISRDNLKNTVYLQMNSLKPEDTALYYCNAEVKVIIWDSRFNYYWGQGTQVTVSS

SEQ ID NO: 2 - protein, 122 aa, synthetic construct
  VARIANT 1: note = X is glutamine (Q) or glutamic acid (E)
  VARIANT 3: note = X is glutamine (Q), glutamic acid (E), or lysine (K)
  VARIANT 5: note = X is glutamine (Q), valine (V), or alanine (A)
  VARIANT 6: note = X is glutamine (Q), valine (V), or alanine (A)
  VARIANT 7: note = X is serine (S) or threonine (T)
  VARIANT 10: note = X is glycine (G) or aspartic acid (D)
  VARIANT 11: note = X is leucine (L), serine (S), or cysteine (C)
  VARIANT 13: note = X is glutamine (Q), arginine (R), or glutamic acid (E)
  VARIANT 14: note = X is threonine (T), alanine (A), or proline (P)
  VARIANT 16: note = X is glycine (G), aspartic acid (D), or glutamic acid (E)
  VARIANT 19: note = X is arginine (R), threonine (T), or serine (S)
  VARIANT 23: note = X is alanine (A), valine (V), threonine (T), or lysine (K)
  VARIANT 24: note = X is alanine (A), valine (V), or threonine (T)
  VARIANT 26: note = X is glycine (G), alanine (A), valine (V), or arginine (R)
  VARIANT 27: note = X is serine (S), arginine (R), threonine (T), leucine (L), or aspartic acid (D)
  VARIANT 28: note = X is phenylalanine (F), threonine (T), or isoleucine (I)
  VARIANT 29: note = X is phenylalanine (F), valine (V), leucine (L), or serine (S)
  VARIANT 30: note = X is serine (S), arginine (R), glutamine (Q), or asparagine (N)
  VARIANT 31: note = X is isoleucine (I), serine (S), threonine (T), phenylalanine (F), leucine (L), lysine (K), asparagine (N), glutamic acid (E), or valine (V).
  VARIANT 32: note = X is asparagine (N), tyrosine (Y), isoleucine (I), phenylalanine (F), or glycine (G)
  VARIANT 33: note = X is aspartic acid (D), threonine (T), serine (S), alanine (A), glutamine (Q), arginine (R), or valine (V)
  VARIANT 34: note = X is methionine (M), valine (V), or isoleucine (I)
  VARIANT 35: note = X is glycine (G), arginine (R), threonine (T), tyrosine (Y), alanine (A), or asparagine (N)
  VARIANT 37: note = X is tyrosine (Y) or phenylalanine (F)
  VARIANT 40: note = X is alanine (A) or threonine (T)
  VARIANT 43: note = X is lysine (K), threonine (T), or serine (S)
  VARIANT 44: note = X is glutamine (Q) or glutamic acid (E)
  VARIANT 47: note = X is leucine (L) or phenylalanine (F)
  VARIANT 49: note = X is alanine (A) or serine (S)
  VARIANT 50: note = X is methionine (M), alanine (A), threonine (T), or serine (S)
  VARIANT 51: note = X is isoleucine (I), serine (S), threonine (T), or arginine (R)
  VARIANT 52: note = X is threonine (T), valine (V), glutamine (Q), asparagine (N), or serine (S)
  VARIANT 53: note = X is glycine (G), serine (S), aspartic acid (D), threonine (T), asparagine (N), alanine (A), or tryptophan (W)
  VARIANT 54: note = X is aspartic acid (D), glycine (G), serine (S), or isoleucine (I)
  VARIANT 55: note = X is aspartic acid (D), glycine (G), tyrosine (Y), or arginine (R)
  VARIANT 56: note = X is serine (S), arginine (R), asparagine (N), or alanine (A)
  VARIANT 57: note = X is threonine (T) or isoleucine (I)
  VARIANT 58: note = X is asparagine (N), tyrosine (Y), threonine (T), serine (S), lysine (K), arginine (R), aspartic acid (D), or histidine (H).
  VARIANT 59: note = X is tyrosine (Y)or histidine (H)
  VARIANT 60: note = X is alanine (A), glutamic acid (E), aspartic acid (D), valine (V), isoleucine (I), threonine (T), serine (S), or lysine (K)
  VARIANT 61: note = X is aspartic acid (D) or glutamic acid (E)
  VARIANT 62: note = X is serine (S) or phenylalanine (F)
  VARIANT 63: note = X is valine (V) or alanine (A)
  VARIANT 64: note = X is lysine (K), threonine (T), or glutamic acid (E)
  VARIANT 65: note = X is glycine (G) or aspartic acid (D)
  VARIANT 67: note = X is phenylalanine (F) or valine (V)
  VARIANT 69: note = X is isoleucine (I) or alanine (A)
  VARIANT 68: note = X is threonine (T) or alanine (A)
  VARIANT 71: note = X is arginine (R) or glycine (G)
  VARIANT 73: note = X is asparagine (N), threonine (T), phenylalanine (F), or aspartic acid (D),
  VARIANT 74: note = X is leucine (L), alanine (A), valine (V), methionine (M), serine (S), or aspartic acid (D)
  VARIANT 75: note = X is lysine (K), glutamine (Q), or glutamic acid (E)
  VARIANT 76: note = X is asparagine (N), threonine (T), glycine (G), histidine (H), or alanine (A)
  VARIANT 77: note = X is threonine (T), leucine (L), or isoleucine (I)
  VARIANT 78: note = X is valine (V) or aspartic acid (D)
  VARIANT 79: note = X is tyrosine (Y), serine (S), histidine (H), or aspartic acid (D)
  VARIANT 81: note = X is glutamine (Q), aspartic acid (D), or glutamic acid (E)
  VARIANT 82: note = X is methionine (M), leucine (L), or valine (V)
  VARIANT 83: note = X is asparagine (N), serine (S), or threonine (T)
  VARIANT 84: note = X is serine (S), aspartic acid (D), or asparagine (N)
  VARIANT 86: note = X is lysine (K) or glutamic acid (E)
  VARIANT 87: note = X is proline (P), valine (V), or serine (S),
  VARIANT 88: note = X is glutamic acid (E), lysine (K), or aspartic acid (D)
  VARIANT 92: note = X is leucine (L) or valine (V)
  VARIANT 94: note = X is tyrosine (Y), arginine (R), or threonine (T)
  VARIANT 96: note = X is asparagine (N), alanine (A), histidine (H), lysine (K), valine (V), or threonine (T)
  VARIANT 97: note = X is alanine (A), arginine (R), tyrosine (Y), proline (P), lysine (K), valine (V), or threonine (T)
  VARIANT 98: note = X is glutamic acid (E), aspartic acid (D), alanine (A), threonine (T), lysine (K), arginine (R), or serine (S)
  VARIANT 99: note = X is valine (V), arginine (R), glycine (G), lysine (K), proline (P), leucine (L), aspartic acid (D), or phenylalanine (F)
  VARIANT 100: note = X is lysine (K), glycine (G), tyrosine (Y), serine (S), arginine (R), aspartic acid (D), or glutamic acid (E)
  VARIANT 101: note = X is valine (V), tyrosine (Y), phenylalanine (F), tryptophan (W), glycine (G), isoleucine (I), or proline (P)
  VARIANT 102: note = X is isoleucine (I), valine (V), tyrosine (Y), phenylalanine (F), serine (S), glutamine (Q), or arginine (R)
  VARIANT 103: note = X is isoleucine (I), valine (V), leucine (L), serine (S), threonine (T), tyrosine (Y), or cysteine (C)
  VARIANT 104: note = X is tryptophan (W), tyrosine (Y), phenylalanine (F), leucine (L), valine (V), threonine (T), or proline (P)
  VARIANT 105: note = X is aspartic acid (D), arginine (R), alanine (A), serine (S), arginine (R), proline (P), or cysteine (C)
  VARIANT 106: note = X is serine (S), alanine (A), threonine (T), proline (P), cysteine (C), glutamine (Q), aspartic acid (D), or tryptophan (W)
  VARIANT 107: note = X is arginine (R), lysine (K), aspartic acid (D), glutamic acid (E), threonine (T), proline (P), or tyrosine (Y).
  VARIANT 108: note = X is phenylalanine (F), aspartic acid (D), tryptophan (W), lysine (K), tyrosine (Y), or serine (S).
  VARIANT 109: note = X is asparagine (N), serine (S), aspartic acid (D), glutamic acid (E), phenylalanine (F), or tyrosine (Y)
  VARIANT 110: note = X is tyrosine (Y), aspartic acid (D), asparagine (N), glutamic acid (E), alanine (A), or glycine (G).
  VARIANT 111: note = X is tyrosine (Y), glutamic acid (E), valine (V), serine (S), or threonine (T)
  VARIANT 114: note = X is glutamine (Q) or lysine (K)
  VARIANT 117: note = X is glutamine (Q), glutamic acid (E), arginine (R), leucine (L), or proline (P)
XVXLXXXGGXXVXXGXSLXLSCXXSXXXXXXXXXXWXRQXPGXXREXVXXXXXXXXXXXXXXXXXRXXXSXDXXXXXXXLXXXXLXXXDTAXYXCXXXXXXXXXXXXXXXXWGXGTXVTVSS

SEQ ID NO: 3 - protein, 361 aa, synthetic construct
QVQLQESGGGLVQTGGSLRLSCAASGSFFSINDMGWYRQAPGKQRELVAMITGDDSTNYADSVKGRFTISRDNLKNTVYLQMNSLKPEDTALYYCNAEVKVIIWDSRFNYYWGQGTQVTVSSSGGGGDGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGS

SEQ ID NO: 4 - protein, 361 aa, synthetic construct
  VARIANT 1: note = X is glutamine (Q) or glutamic acid (E)
  VARIANT 3: note = X is glutamine (Q), glutamic acid (E), or lysine (K)
  VARIANT 5: note = X is glutamine (Q), valine (V), or alanine (A)
  VARIANT 6: note = X is glutamine (Q), valine (V), or alanine (A)
  VARIANT 7: note = X is serine (S) or threonine (T)
  VARIANT 10: note = X is glycine (G) or aspartic acid (D)
  VARIANT 11: note = X is leucine (L), serine (S), or cysteine (C)
  VARIANT 13: note = X is glutamine (Q), arginine (R), or glutamic acid (E)
  VARIANT 14: note = X is threonine (T), alanine (A), or proline (P)
  VARIANT 16: note = X is glycine (G), aspartic acid (D), or glutamic acid (E)
  VARIANT 19: note = X is arginine (R), threonine (T), or serine (S)
  VARIANT 23: note = X is alanine (A), valine (V), threonine (T), or lysine (K)
  VARIANT 24: note = X is alanine (A), valine (V), or threonine (T)
  VARIANT 26: note = X is glycine (G), alanine (A), valine (V), or arginine (R)
  VARIANT 27: note = X is serine (S), arginine (R), threonine (T), leucine (L), or aspartic acid (D)
  VARIANT 28: note = X is phenylalanine (F), threonine (T), or isoleucine (I)
  VARIANT 29: note = X is phenylalanine (F), valine (V), leucine (L), or serine (S)
  VARIANT 30: note = X is serine (S), arginine (R), glutamine (Q), or asparagine (N)
  VARIANT 31: note = X is isoleucine (I), serine (S), threonine (T), phenylalanine (F), leucine (L), lysine (K), asparagine (N), glutamic acid (E), or valine (V)
  VARIANT 32: note = X is asparagine (N), tyrosine (Y), isoleucine (I), phenylalanine (F), or glycine (G)
  VARIANT 33: note = X is aspartic acid (D), threonine (T), serine (S), alanine (A), glutamine (Q), arginine (R), or valine (V)
  VARIANT 34: note = X is methionine (M), valine (V), or isoleucine (I)
  VARIANT 35: note = X is glycine (G), arginine (R), threonine (T), tyrosine (Y), alanine (A), or asparagine (N)
  VARIANT 37: note = X is tyrosine (Y) or phenylalanine (F)
  VARIANT 40: note = X is alanine (A) or threonine (T)
  VARIANT 43: note = X is lysine (K), threonine (T), or serine (S)
  VARIANT 44: note = X is glutamine (Q) or glutamic acid (E)
  VARIANT 47: note = X is leucine (L) or phenylalanine (F)
  VARIANT 49: note = X is alanine (A) or serine (S)
  VARIANT 50: note = X is methionine (M), alanine (A), threonine (T), or serine (S)
  VARIANT 51: note = X is isoleucine (I), serine (S), threonine (T), or arginine (R)
  VARIANT 52: note = X is threonine (T), valine (V), glutamine (Q), asparagine (N), or serine (S)
  VARIANT 53: note = X is glycine (G), serine (S), aspartic acid (D), threonine (T), asparagine (N), alanine (A), or tryptophan (W)
  VARIANT 54: note = X is aspartic acid (D), glycine (G), serine (S), or isoleucine (I)
  VARIANT 55: note = X is aspartic acid (D), glycine (G), tyrosine (Y), or arginine (R)
  VARIANT 56: note = X is serine (S), arginine (R), asparagine (N), or alanine (A)
  VARIANT 57: note = X is threonine (T) or isoleucine (I)
  VARIANT 58: note = X is asparagine (N), tyrosine (Y), aspartic acid (D), threonine (T), serine (S), lysine (K), arginine (R), aspartic acid (D), or histidine (H)
  VARIANT 59: note = X is tyrosine (Y)or histidine (H)
  VARIANT 60: note = X is alanine (A), glutamic acid (E), aspartic acid (D), valine (V), isoleucine (I), threonine (T), serine (S), or lysine (K)
  VARIANT 61: note = X is aspartic acid (D) or glutamic acid (E)
  VARIANT 62: note = X is serine (S) or phenylalanine (F)
  VARIANT 63: note = X is valine (V) or alanine (A)
  VARIANT 64: note = X is lysine (K), threonine (T), or glutamic acid (E)
  VARIANT 65: note = X is glycine (G) or aspartic acid (D)
  VARIANT 67: note = X is phenylalanine (F) or valine (V)
  VARIANT 69: note = X is isoleucine (I) or alanine (A)
  VARIANT 68: note = X is threonine (T) or alanine (A)
  VARIANT 71: note = X is arginine (R) or glycine (G)
  VARIANT 73: note = X is asparagine (N), threonine (T), phenylalanine (F), or aspartic acid (D),
  VARIANT 74: note = X is leucine (L), alanine (A), valine (V), methionine (M), serine (S), or aspartic acid (D)
  VARIANT 75: note = X is lysine (K), glutamine (Q), or glutamic acid (E)
  VARIANT 76: note = X is asparagine (N), threonine (T), glycine (G), histidine (H), or alanine (A)
  VARIANT 77: note = X is threonine (T), leucine (L), or isoleucine (I)
  VARIANT 78: note = X is valine (V) or aspartic acid (D)
  VARIANT 79: note = X is tyrosine (Y), serine (S), histidine (H), or aspartic acid (D)
  VARIANT 81: note = X is glutamine (Q), aspartic acid (D), or glutamic acid (E)
  VARIANT 82: note = X is methionine (M), leucine (L), or valine (V)
  VARIANT 83: note = X is asparagine (N), serine (S), or threonine (T)
  VARIANT 84: note = X is serine (S), aspartic acid (D), or asparagine (N)
  VARIANT 86: note = X is lysine (K) or glutamic acid (E)
  VARIANT 87: note = X is proline (P), valine (V), or serine (S),
  VARIANT 88: note = X is glutamic acid (E), lysine (K), or aspartic acid (D)
  VARIANT 92: note = X is leucine (L) or valine (V)
  VARIANT 94: note = X is tyrosine (Y), arginine (R), or threonine (T)
  VARIANT 96: note = X is asparagine (N), alanine (A), histidine (H), lysine (K), valine (V), or threonine (T)
  VARIANT 97: note = X is alanine (A), arginine (R), tyrosine (Y), proline (P), lysine (K), valine (V), or threonine (T)
  VARIANT 98: note = X is glutamic acid (E), aspartic acid (D), alanine (A), threonine (T), lysine (K), arginine (R), or serine (S)
  VARIANT 99: note = X is valine (V), arginine (R), glycine (G), lysine (K), proline (P), leucine (L), aspartic acid (D), or phenylalanine (F)
  VARIANT 100: note = X is lysine (K), glycine (G), tyrosine (Y), serine (S), arginine (R), aspartic acid (D), or glutamic acid (E)
  VARIANT 101: note = X is valine (V), tyrosine (Y), phenylalanine (F), tryptophan (W), glycine (G), isoleucine (I), or proline (P)
  VARIANT 102: note = X is isoleucine (I), valine (V), tyrosine (Y), phenylalanine (F), serine (S), glutamine (Q), or arginine (R)
  VARIANT 103: note = X is isoleucine (I), valine (V), leucine (L), serine (S), threonine (T), tyrosine (Y), or cysteine (C)
  VARIANT 104: note = X is tryptophan (W), tyrosine (Y), phenylalanine (F), leucine (L), valine (V), threonine (T), or proline (P)
  VARIANT 105: note = X is aspartic acid (D), arginine (R), alanine (A), proline (P), serine (S), proline (P), or cysteine (C)
  VARIANT 106: note = X is serine (S), alanine (A), threonine (T), proline (P), cysteine (C), glutamine (Q), aspartic acid (D), or tryptophan (W)
  VARIANT 107: note = X is arginine (R), lysine (K), aspartic acid (D), glutamic acid (E), threonine (T), proline (P), or tyrosine (Y)
  VARIANT 108: note = X is phenylalanine (F), aspartic acid (D), tryptophan (W), lysine (K), tyrosine (Y), or serine (S).
  VARIANT 109: note = X is asparagine (N), serine (S), aspartic acid (D), glutamic acid (E), phenylalanine (F), or tyrosine (Y)
  VARIANT 110: note = X is tyrosine (Y), aspartic acid (D), glutamic acid (E), asparagine (N), alanine (A), or glycine (G)
  VARIANT 111: note = X is tyrosine (Y), glutamic acid (E), valine (V), serine (S), or threonine (T)
  VARIANT 114: note = X is glutamine (Q) or lysine (K)
  VARIANT 117: note = X is glutamine (Q), glutamic acid (E), arginine (R), leucine (L), or proline (P)
XVXLXXXGGXXVXXGXSLXLSCXXSXXXXXXXXXXWXRQXPGXXREXVXXXXXXXXXXXXXXXXXRXXXSXDXXXXXXXLXXXXLXXXDTAXYXCXXXXXXXXXXXXXXXXWGXGTXVTVSSSGGGGDGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREELTKNQVSLTCLVKGFYPSDIAVEWESNGQPENNYKTTPPVLDSDGSFFLYSKLTVDKSRWQQGNVFSCSVMHEALHNHYTQKSLSLSPGS

SEQ ID NO: 5 - protein, 7 aa, synthetic construct
GSFFSIN

SEQ ID NO: 6 - protein, 5 aa, synthetic construct
INDMG

SEQ ID NO: 7 - protein, 8 aa, synthetic construct
GSFFSIND

SEQ ID NO: 8 - protein, 7 aa, synthetic construct
  VARIANT 1: note = The residue at this position can be glycine (G), as shown, or, alternatively, one of the following residues: alanine (A), valine (V), or arginine (R)
  VARIANT 2: note = The residue at this position can be serine (S), as shown, or, alternatively, one of the following residues: arginine (R), threonine (T), leucine (L), or aspartic acid (D).
  VARIANT 3: note = The residue at this position can be phenylalanine (F), as shown, or, alternatively, one of the following residues: threonine (T), or isoleucine (I).
  VARIANT 4: note = The residue at this position can be phenylalanine (F) as shown, or, alternatively, one of the following residues: valine (V), leucine (L), or serine (S).
  VARIANT 5: note = The residue at this position can be serine (S) as shown, or, alternatively, one of the following residues: arginine (R), glutamine (Q), or asparagine (N).
  VARIANT 6: note = The residue at this position can be isoleucine (I), as shown, or, alternatively, one of the following residues: serine (S), threonine (T), phenylalanine (F), leucine (L), lysine (K), asparagine (N), glutamic acid (E), or valine (V).
  VARIANT 7: note = The residue at this position can be asparagine (N), as shown, or, alternatively, one of the following residues: tyrosine (Y), isoleucine (I), phenylalanine (F), or glycine (G).
GSFFSIN

SEQ ID NO: 9 - protein, 5 aa, synthetic construct
  VARIANT 1: note = The residue at this position can be isoleucine (I) as shown, or, alternatively, one of the following residues: serine (S), threonine (T), phenylalanine (F), leucine (L), lysine (K), asparagine (N), glutamic acid (E), or valine (V).
  VARIANT 2: note = The residue at this position can be asparagine (N) as shown, or, alternatively, one of the following residues: tyrosine (Y), isoleucine (I), phenylalanine (F), or glycine (G).
  VARIANT 3: note = The residue at this position can be aspartic acid (D) as shown, or, alternatively, one of the following residues: threonine (T), serine (S), alanine (A), glutamine (Q), arginine (R), or valine (V).
  VARIANT 4: note = The residue at this position can be methionine (M) as shown, or, alternatively, one of the following residues: valine (V), or isoleucine (I).
  VARIANT 5: note = The residue at this position can be glycine (G) as shown, or, alternatively, one of the following residues: arginine (R), threonine (T), tyrosine (Y), alanine (A), or asparagine (N).
IDNGM

SEQ ID NO: 10 - protein, 8 aa, synthetic construct
  VARIANT 1: note = The residue at this position can be glycine (G) as shown, or, alternatively, one of the following residues: alanine (A), valine (V), or arginine (R).
  VARIANT 2: note = The residue at this position can be serine (S) as shown, or, alternatively, one of the following residues: arginine (R), threonine (T), leucine (L), or aspartic acid (D).
  VARIANT 3: note = The residue at this position can be phenylalanine (F) as shown, or, alternatively, one of the following residues: threonine (T), or isoleucine (I).
  VARIANT 4: note = The residue at this position can be phenylalanine (F) as shown, or, alternatively, one of the following residues: valine (V), leucine (L), or serine (S).
  VARIANT 5: note = X is serine (S), arginine (R), glutamine (Q), or asparagine (N)
  VARIANT 6: note = X is isoleucine (I), serine (S), threonine (T), phenylalanine (F), leucine (L), lysine (K), asparagine (N), glutamic acid (E), or valine (V).
  VARIANT 7: note = X is asparagine (N), tyrosine (Y), isoleucine (I), phenylalanine (F), or glycine (G)
  VARIANT 8: note = X is aspartic acid (D), threonine (T), serine (S), alanine (A), glutamine (Q), arginine (R), or valine (V)
GSFFXXXX

SEQ ID NO: 11 - protein, 5 aa, synthetic construct
TGDDS

SEQ ID NO: 12 - protein, 16 aa, synthetic construct
MITGDDSTNYADSVKG

SEQ ID NO: 13 - protein, 7 aa, synthetic construct
ITGDDST

SEQ ID NO: 14 - protein, 5 aa, synthetic construct
  VARIANT 1: note = The residue at this position can be threonine (T) as shown, or, alternatively, one of the following residues: valine (V), glutamine (Q), asparagine (N), or serine (S)
  VARIANT 2: note = The residue at this position can be glycine (G) as shown, or, alternatively, one of the following residues: serine (S), aspartic acid (D), threonine (T), asparagine (N), alanine (A), or tryptophan (W).
  VARIANT 3: note = The residue at this position can be aspartic acid (D) as shown, or, alternatively, one of the following residues: glycine (G), serine (S), or isoleucine (I).
  VARIANT 4: note = The residue at this position can be aspartic acid (D) as shown, or, alternatively, one of the following residues: glycine (G), tyrosine (Y), or arginine (R).
  VARIANT 5: note = The residue at this position can be serine (S) as shown, or, alternatively, one of the following residues: arginine (R), asparagine (N), or alanine (A).
TGDDS

SEQ ID NO: 15 - protein, 16 aa, synthetic construct
  VARIANT 1: note = The residue at this position can be methionine (M) as shown, or, alternatively, one of the following residues: alanine (A), threonine (T), or serine (S).
  VARIANT 2: note = The residue at this position can be isoleucine (I) as shown, or, alternatively, one of the following residues: serine (S), threonine (T), or arginine (R).
  VARIANT 3: note = The residue at this position can be threonine (T) as shown, or, alternatively, one of the following residues: valine (V), glutamine (Q), asparagine (N), or serine (S).
  VARIANT 4: note = The residue at this position can be glycine (G) as shown, or, alternatively, one of the following residues: serine (S), aspartic acid (D), threonine (T), asparagine (N), alanine (A), or tryptophan (W).
  VARIANT 5: note = X is aspartic acid (D), glycine (G), serine (S), or isoleucine (I)
  VARIANT 6: note = X is aspartic acid (D), glycine (G), tyrosine (Y), or arginine (R)
  VARIANT 7: note = X is serine (S), arginine (R), asparagine (N), or alanine (A)
  VARIANT 8: note = X is threonine (T) or isoleucine (I)
  VARIANT 9: note = X is asparagine (N), tyrosine (Y), threonine (T), serine (S), lysine (K), arginine (R), aspartic acid (D), or histidine (H)
  VARIANT 10: note = X is tyrosine (Y) or histidine (H)
  VARIANT 11: note = X is alanine (A), glutamic acid (E), aspartic acid (D), valine (V), isoleucine (I), threonine (T), serine (S), or lysine (K)
  VARIANT 12: note = X is aspartic acid (D) or glutamic acid (E)
  VARIANT 13: note = X is serine (S) or phenylalanine (F)
  VARIANT 14: note = X is valine (V) or alanine (A)
  VARIANT 15: note = X is lysine (K), threonine (T), or glutamic acid (E)
  VARIANT 16: note = X is glycine (G) or aspartic acid (D)
MITGXXXXXXXXXXXX

SEQ ID NO: 16 - protein, 7 aa, synthetic construct
  VARIANT 1: note = The residue at this position can be isoleucine (I) as shown, or, alternatively, one of the following residues: serine (S), threonine (T), or arginine (R).
  VARIANT 2: note = The residue at this position can be threonine (T) as shown, or, alternatively, one of the following residues: valine (V), glutamine (Q), asparagine (N), or serine (S).
  VARIANT 3: note = The residue at this position can be glycine (G) as shown, or, alternatively, one of the following residues: serine (S), aspartic acid (D), threonine (T), asparagine (N), alanine (A), or tryptophan (W).
  VARIANT 4: note = The residue at this position can be aspartic acid (D) as shown, or, alternatively, one of the following residues: glycine (G), serine (S), or isoleucine (I).
  VARIANT 5: note = X is aspartic acid (D), glycine (G), tyrosine (Y), or arginine (R)
  VARIANT 6: note = X is serine (S), arginine (R), asparagine (N), or alanine (A)
  VARIANT 7: note = X is threonine (T) or isoleucine (I)
ITGDXXX

SEQ ID NO: 17 - protein, 14 aa, synthetic construct
EVKVIIWDSRFNYY

SEQ ID NO: 18 - protein, 16 aa, synthetic construct
NAEVKVIIWDSRFNYY

SEQ ID NO: 19 - protein, 14 aa, synthetic construct
  VARIANT 1: note = The residue at this position can be glutamic acid (E) as shown, or, alternatively, one of the following residues: aspartic acid (D), alanine (A), threonine (T), lysine (K), arginine (R), or serine (S).
  VARIANT 2: note = The residue at this position can be valine (V) as shown, or, alternatively, one of the following residues: arginine (R), glycine (G), lysine (K), proline (P), leucine (L), aspartic acid (D), or phenylalanine (F).
  VARIANT 3: note = The residue at this position can be lysine (K) as shown, or, alternatively, one of the following residues: glycine (G), tyrosine (Y), serine (S), arginine (R), aspartic acid (D), or glutamic acid (E).
  VARIANT 4: note = The residue at this position can be valine (V) as shown, or, alternatively, one of the following residues: tyrosine (Y), phenylalanine (F), tryptophan (W), glycine (G), isoleucine (I), or proline (P).
  VARIANT 5: note = X is isoleucine (I), valine (V), tyrosine (Y), phenylalanine (F), serine (S), glutamine (Q), or arginine (R)
  VARIANT 6: note = X is isoleucine (I), valine (V), leucine (L), serine (S), threonine (T), tyrosine (Y), or cysteine (C)
  VARIANT 7: note = X is tryptophan (W), tyrosine (Y), phenylalanine (F), leucine (L), valine (V), threonine (T), or proline (P)
  VARIANT 8: note = X is aspartic acid (D), arginine (R), alanine (A), proline (P), serine (S), arginine (R), proline (P), or cysteine (C)
  VARIANT 9: note = X is serine (S), alanine (A), threonine (T), proline (P), cysteine (C), glutamine (Q), aspartic acid (D), or tryptophan (W)
  VARIANT 10: note = X is arginine (R), lysine (K), aspartic acid (D), glutamic acid (E), threonine (T), proline (P), tyrosine (Y), or arginine (R)
  VARIANT 11: note = X is phenylalanine (F), aspartic acid (D), tryptophan (W), lysine (K), tyrosine (Y), serine (S), or phenylalanine (F)
  VARIANT 12: note = X is asparagine (N), serine (S), aspartic acid (D), glutamic acid (E), phenylalanine (F), or tyrosine (Y)
  VARIANT 13: note = X is tyrosine (Y), aspartic acid (D), glutamic acid (E), asparagine (N), alanine (A), or glycine (G)
  VARIANT 14: note = X is tyrosine (Y), glutamic acid (E), valine (V), serine (S), or threonine (T)
EVKVXXXXXXXXXX

SEQ ID NO: 20 - protein, 16 aa, synthetic construct
  VARIANT 1: note = The residue at this position can be asparagine (N), as shown, or, alternatively, one of the following residues: alanine (A), histidine (H), lysine (K), valine (V), or threonine (T).
  VARIANT 2: note = The residue at this position can be alanine (A) as shown, or, alternatively, one of the following residues: arginine (R), tyrosine (Y), proline (P), lysine (K), valine (V), or threonine (T).
  VARIANT 3: note = The residue at this position can be glutamic acid (E), as shown, or, alternatively, one of the following residues: aspartic acid (D), alanine (A), threonine (T), lysine (K), arginine (R), or serine (S).
  VARIANT 4: note = The residue at this position can be valine (V) as shown, or, alternatively, one of the following residues: arginine (R), glycine (G), lysine (K), proline (P), leucine (L), aspartic acid (D), or phenylalanine (F).
  VARIANT 5: note = X is lysine (K), glycine (G), tyrosine (Y), serine (S), arginine (R), aspartic acid (D), or glutamic acid (E)
  VARIANT 6: note = X is valine (V), tyrosine (Y), phenylalanine (F), tryptophan (W), glycine (G), valine (V), isoleucine (I), or proline (P)
  VARIANT 7: note = X is isoleucine (I), valine (V), tyrosine (Y), phenylalanine (F), serine (S), glutamine (Q), or arginine (R)
  VARIANT 8: note = X is isoleucine (I), valine (V), leucine (L), serine (S), threonine (T), tyrosine (Y), or cysteine (C)
  VARIANT 9: note = X is tryptophan (W), tyrosine (Y), phenylalanine (F), leucine (L), valine (V), threonine (T), or proline (P)
  VARIANT 10: note = X is aspartic acid (D), arginine (R), alanine (A), proline (P), serine (S), arginine (R), or cysteine (C)
  VARIANT 11: note = X is serine (S), alanine (A), threonine (T), proline (P), cysteine (C), glutamine (Q), aspartic acid (D), or tryptophan (W)
  VARIANT 12: note = X is arginine (R), lysine (K), aspartic acid (D), glutamic acid (E), threonine (T), proline (P), or tyrosine (Y).
  VARIANT 13: note = X is phenylalanine (F), aspartic acid (D), tryptophan (W), lysine (K), tyrosine (Y), serine (S), or phenylalanine (F)
  VARIANT 14: note = X is asparagine (N), serine (S), aspartic acid (D), glutamic acid (E), phenylalanine (F), or tyrosine (Y)
  VARIANT 15: note = X is tyrosine (Y), aspartic acid (D), glutamic acid (E), asparagine (N), alanine (A), or glycine (G)
  VARIANT 16: note = X is tyrosine (Y), glutamic acid (E), valine (V), serine (S), or threonine (T)
NAEVXXXXXXXXXXXX

SEQ ID NO: 21 - protein, 207 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNK

(19) United States
(12) Patent Application Publication
(10) Pub. No.: US 2026/0356921 A1
(43) Pub. Date: Nov. 12, 2026

(54) SARS-COV-2 BINDING AGENTS AND USES THEREOF

(71) Applicant: Boost Biopharma, Inc., Woburn, MA (US)

(72) Inventors: Fritz M. SCHOMBURG, Woburn, MA (US); David M. RANCOUR, Woburn, MA (US)

(73) Assignee: Boost Biopharma, Inc., Woburn, MA (US)

(21) Appl. No.: 19/108,047

(22) PCT Filed: Aug. 31, 2023
(86) PCT No.: PCT/US2023/073213

§ 371 Date: Feb. 28, 2025

Related U.S. Application Data
(60) Provisional application No. 63/402,839, filed on Aug. 31, 2022.

(51) Int. Cl. C07K 16/104; A61K 39/00

(52) U.S. Cl. CPC C07K 16/104; A61K 2039/54; C07K 2317/24; C07K 2317/31; C07K 2317/52; C07K 2317/565; C07K 2317/569; C07K 2317/76; C07K 2317/92

(57) ABSTRACT

The invention relates to SARS-CoV-2 binding agents and uses thereof.

### CROSS-REFERENCE TO PRIOR APPLICATION

This application claims benefit to U.S. Provisional Patent Application No. 63/402,839, filed Aug. 31, 2022, which is hereby incorporated by reference in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 128,382 Byte XML file named 768120.xml, created Aug. 31, 2023.

### BACKGROUND OF THE INVENTION

The rapid evolution of new severe acute respiratory syndrome coronavirus 2 (SARS-CoV-2) variants containing mutations that alter the amino acid sequence of the Spike protein have resulted in variants with altered function, resistance to native immune defenses, and resistance to immune defenses elicited by currently marketed vaccines and treatments, such as monoclonal antibody treatments.

There remains a significant unmet medical need for effective treatments and/or prophylactic agents against infection with recently-arising variants of SARS-CoV-2, including through the use of monoclonal antibodies.

### BRIEF SUMMARY OF THE INVENTION

Provided herein is a SARS-CoV-2 binding agent, and compositions and kits comprising the same. Also provided are methods for preventing or treating a disease, disorder, or condition in a mammal that is responsive to inhibition of SARS-CoV-2 binding to an ACE2 receptor or for enhancing an immune response in a mammal by administering to the mammal the SARS-CoV-2 binding agent. The disclosure also provides a method of detecting SARS-CoV-2 in a sample. The disclosure further provides a method for providing a SARS-CoV-2 binding agent comprising expressing in a cell in vitro or in vivo one or more nucleic acids encoding the SARS-CoV-2 binding agent. These and other aspects of the invention will be apparent to the skilled person reading the detailed description.

### BRIEF DESCRIPTION OF THE DRAWINGS

FIG. 1A is a schematic depicting the binding of SARS-CoV-2 receptor binding domain (RBD) to an exemplary SARS-CoV-2 binding agent of the invention.

FIG. 1B is a schematic depicting the binding of SARS-CoV-2 receptor binding domain (RBD) to an exemplary SARS-CoV-2 binding agent of the invention.

FIG. 2 is a schematic depicting XBB.1.5 RBD Amino Acid Sequence with Predicted Contact Sites (CS) for exemplary SARS-CoV-2 binding agent of the invention. Sites highlighted with black boxes are conserved across all SARS-CoV-2 variants and original SARS-CoV-2003 and thought to be primary determinants for binding interaction between the binding agent and the SARS-CoV-2 variants. Sites highlighted with white boxes exhibit some variation in sequence between SARS-COV-2 variants. Lines connecting cysteine (C) amino acids represent intramolecular disulfide bonds.

FIG. 3 is a chart showing the alignment of certain SARS-CoV RBD sequences.

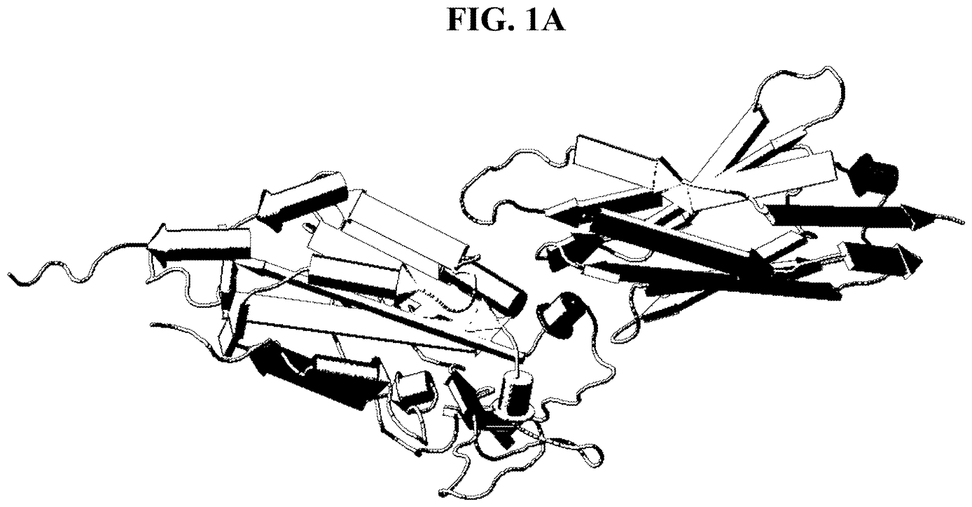

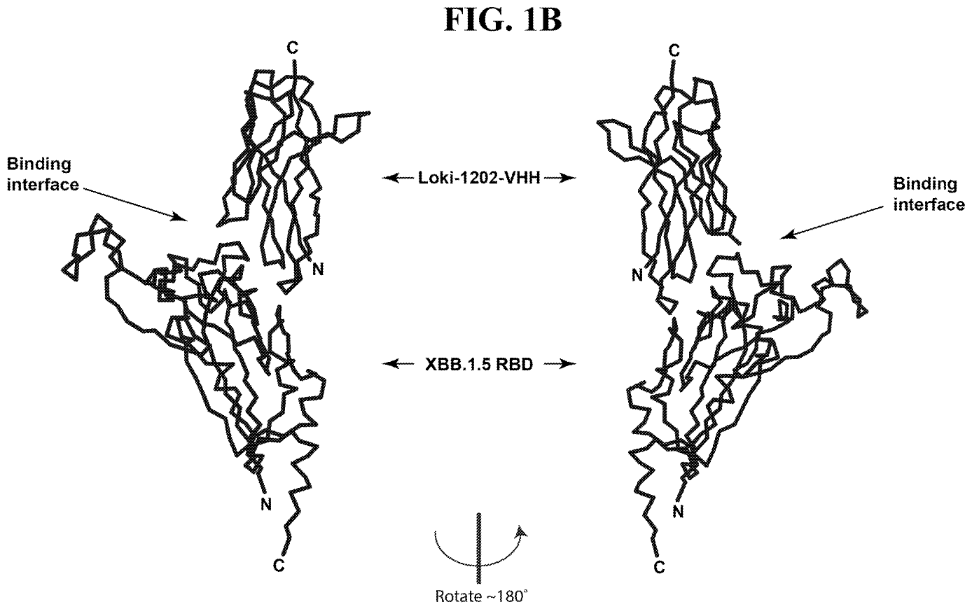

### DETAILED DESCRIPTION OF THE INVENTION

### I. Single Domain Antibodies and Antibody Fragments

The severe acute respiratory syndrome coronavirus 2 (SARS-CoV-2) binding agent is any suitable agent that specifically binds SARS-CoV-2. In some embodiments, the severe acute respiratory syndrome coronavirus 2 (SARS-CoV-2) binding agent comprises, consists of, or consists essentially of a single domain antibody or single domain antibody fragment. The terms “single domain antibody” and “single domain antibody fragment,” as used in this disclosure, refer to polypeptides that comprise, consist of, or consist essentially of at least one an antibody single variable domain region (e.g., a VH or VHH, also known as a nanobody) that specifically binds an antigen or epitope independently of other variable regions or domains. In other words, the single variable domain alone forms the antigen binding site of the antibody, without interaction with another variable domain (e.g., a light chain variable domain). In some embodiments, the antibody single variable domain is a single heavy chain variable domain, such as a VHH domain, humanized VHH domain and/or camelized VH domains such as camelized human VH domains. In some embodiments, the single domain antibody and fragments thereof are heavy-chain only antibodies, or at least the variable region thereof or a portion thereof. In some embodiments, the SARS-CoV-2 binding agent that comprises, consists of, or consists essentially of a single domain antibody or single domain antibody fragment does not include a light chain variable region.

A single domain antibody or single domain antibody fragment generally comprises three complementarity determining regions (CDRs), i.e., CDR1, CDR2, and CDR3. However, in some instances, a single domain antibody or single domain antibody fragment comprising only one CDR, e.g., CDR3, has been found to specifically bind an antigen. See, for example, Qiu et al., A camel anti-lysozyme CDR3 only domain antibody selected from phage display VHH library acts as potent lysozyme inhibitor, Acta Biochimica et Biophysica Sinica, 49(6): 513-519 (2017). The CDRs of a given Ig sequence—be it of a single domain antibody or a canonical antibody—can be determined by any of several conventional numbering schemes, such as Kabat, Chothia, Martin (Enhanced Chothia), IGMT, or AHo (see, e.g., Kabat, et al., Sequences of Proteins of Immunological Interest, U.S. Department of Health and Human Services, NIH (1991); Chothia, et al., Canonical Structures for the Hypervariable Regions of Immunoglobulins, J. Mol. Biol., 196: 901-917 (1987); Al-Lazikani et al., Standard Conformations for the Canonical Structures of Immunoglobulins, J. Mol. Biol., 273: 927-948 (1997); Abhinandan et al., Analysis and Improvements to Kabat and Structurally Correct Numbering of Antibody Variable Domains, Mol. Immunol., 45: 3832-3839 (2008); Lefranc et al., The IMGT unique numbering for immunoglobulins, T cell Receptors and Ig-like domains, The Immunologist, 7: 132-136 (1999); Lefranc et al., IMGT unique numbering for immunoglobulin and T cell receptor variable domains and I superfamily V-like domains, Dev. Comp. Immunol., 27: 55-77 (2003); and Honegger et al., Yet another numbering scheme for immunoglobulin variable domains: an automatic modeling and analysis tool, J. Mol. Biol., 309: 657-670 (2001).

In some embodiments, the antibody single variable domain is a human antibody variable domain. In other embodiments, the antibody single variable domain is camelid (e.g., camel, llama, alpaca, dromedary and guanaco), chondrichthian (e.g., shark (e.g., nurse shark), skates, or rays), rodent, or human, or is derived therefrom. In some embodiments, the antibody single variable domain—or the single domain antibody or fragment thereof that comprises, consists of, or consists essentially of the antibody single variable domain—is species optimized (e.g., humanized). In some embodiments, a humanized antibody single variable domain has been modified to remove and/or modify residues that facilitate binding between heavy and light chain immunoglobulin polypeptides.

In some embodiments, antibody single variable domains are present in a format (e.g., homo- or hetero-multimer) with other variable regions or variable domains where the other regions or domains are not required for antigen binding by the single variable domain (i.e., where the single variable domain binds antigen independently of the additional variable domains). For instance, in some embodiments, the SARS-CoV-2 binding agent comprises, consists of, or consists essentially of 1, 2, 3, 4, 5, 6, 7, 8, 9 or 10 antibody single variable domains, preferably 1, 2, 3 or 4 such domains, more preferably 1, 2 or 3 such domains, more preferably 1 or 2 such domains, or yet more preferably 1 such domain. In some embodiments wherein the SARS-CoV-2 binding agent comprises, consists of, or consists essentially of a plurality of antibody single variable domains, all of the domains are the same or substantially the same. In some embodiments wherein the SARS-CoV-2 binding agent comprises, consists of, or consists essentially of a plurality of antibody single variable domains, none of the domains are the same. In further embodiments, some, but not all, of the antibody single variable domains are the same or substantially the same. In some embodiments, the SARS-CoV-2 binding agent comprises, consists of, or consists essentially of at least one antibody single variable domain (.e.g, VHH) and at least one other antibody or antibody fragment (e.g., any combination of a Fab fragment, a F(ab′)2 fragment, an Fv fragment, a Fab′ fragment, a dsFv, or an scFv). In some embodiments, the at least one antibody single variable domain and the at least one other antibody or antibody fragment specifically bind to different epitopes. In some embodiments, the at least one antibody single variable domain and the at least one other antibody or antibody fragment specifically bind to the same epitope, or the same antigen at different epitopes. In certain embodiments, the SARS-CoV-2 binding agent is a bispecific heavy chain only antibody comprising, consisting of, or consisting essentially of at least one antibody single variable domain that specifically binds to SARS-CoV-2. See, e.g., Suurs, et al., A review of bispecific antibodies and antibody constructs in oncology and clinical challenges, Pharmacology & Therapeutics, 203:103-119 (September 2019) for various constructs that in certain embodiments the SARS-CoV-2 binding agent can comprise, consist of, or consist essentially of.

In some embodiments, the SARS-CoV-2 binding agent comprises, consists of, or consists essentially of at least one antibody single variable domains and at least one antibody Fc region, preferably one antibody Fc region. In some embodiments, the Fc region is human, camel, llama, alpaca, dromedary and guanaco, mouse or rodent, or derived therefrom. In certain embodiments wherein the Fc region is human, the Fc region is IgG1, IgG2, IgG3, or IgG4, preferably IgG1.

In some embodiments, the SARS-CoV-2 binding agent comprising, consisting of, or consisting essentially of at least one antibody single variable domain is an antibody conjugate. In this respect, the SARS-CoV-2 binding agent can be a conjugate of (1) an antibody, an alternative scaffold, or fragments thereof, and (2) a protein or non-protein moiety comprising the SARS-CoV-2 binding agent. For example, the SARS-CoV-2 binding agent can be all or part of an antibody single variable domain conjugated to a peptide (e.g., PEG or serum albumin), a solid support (e.g., a substrate, a surface, a particle), a fluorescent molecule, a molecule or substance suitable for detection, e.g., a radiological agent or an imaging or dye agent or tag, other detection molecules or substances such as gold particles, a pharmaceutical agent, or a radiotherapy agent. Any method known in the art for separately conjugating an antigen-binding agent (e.g., an antibody) to a detectable moiety may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. and Cytochem., 30: 407-412 (1982)).

In some embodiments, the single domain antibody or single domain antibody fragment is used as an intrabody, i.e., expressed intracellularly following gene transfer of a nucleic acid encoding the single domain antibody or single domain antibody fragment.

In some embodiments, the single domain antibody or single domain antibody fragment comprises, consists of, or consists essentially of a single domain variable region that comprises, consists of, or consists essentially of the amino acid sequence of SEQ ID NO: 1, or at least the CDRs thereof. In other embodiments, the single domain variable region comprises, consists of, or consists essentially of the amino acid sequence of SEQ ID NO: 2, or at least the CDRs thereof.

In some embodiments, the single domain variable region comprises, consists of, or consists essentially of the amino acid sequence of SEQ ID NO: 2, wherein the amino acid sequence differs from the amino acid sequence of SEQ ID NO: 1 at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 79 or 80 residues, preferably at only 1, 2, 3, 4, 5, 6, 7, 8, 9, or 10 residues, more preferably at only 1, 2, or 3 residues, even more preferably at only 1 or 2 residues, yet even more preferably at only 1 residue.

In some embodiments, the single domain variable region comprises, consists of, or consists essentially of the amino acid sequence of SEQ ID NO: 1, except that the amino acid sequence differs from the amino acid sequence of SEQ ID NO: 1 at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 79 or 80 residues, preferably at only 1, 2, 3, 4, 5, 6, 7, 8, 9, or 10 residues, more preferably at only 1, 2, or 3 residues, even more preferably at only 1 or 2 residues, yet even more preferably at only 1 residue.

In an embodiment, the single domain variable region comprises, consists of, or consists essentially of the amino acid sequence of SEQ ID NO: 1 or SEQ ID NO: 2, or at least the CDRs thereof. In another embodiment, the single domain variable region comprises, consists of, or consists essentially of the amino acid sequence of SEQ ID NO: 2, or at least the CDRs thereof, with any of the aforementioned amino acid substitutions in any suitable combination.

In some embodiments, the anti-SARS-CoV-2 single domain antibody or single domain antibody fragment comprises: a CDR1 of the single domain variable region (CDR1) comprising, consisting of, or consisting essentially of the amino acid sequence SEQ ID NO: 5, SEQ ID NO: 6, or SEQ ID NO: 7; a CDR2 of the single domain variable region (CDR2) comprising, consisting of, or consisting essentially of the amino acid sequence of SEQ ID NO: 11, SEQ ID NO: 12, or SEQ ID NO: 13; and/or a CDR3 of the single domain variable region (CDR3) comprising, consisting of, or consisting essentially of the amino acid sequence SEQ ID NO: 17 or SEQ ID NO: 18.

In another embodiment, the anti-SARS-CoV-2 single domain antibody or single domain antibody fragment comprises: a CDR1 of the single domain variable region (CDR1) comprising, consisting of, or consisting essentially of the amino acid sequence SEQ ID NO: 5; a CDR2 of the single domain variable region (CDR2) comprising, consisting of, or consisting essentially of the amino acid sequence of SEQ ID NO: 11; and/or a CDR3 of the single domain variable region (CDR3) comprising, consisting of, or consisting essentially of the amino acid sequence SEQ ID NO: 17.

In some embodiments, the anti-SARS-CoV-2 single domain antibody or single domain antibody fragment comprises: a CDR1 of the single domain variable region (CDR1) comprising, consisting of, or consisting essentially of the amino acid sequence SEQ ID NO: 6; a CDR2 of the single domain variable region (CDR2) comprising, consisting of, or consisting essentially of the amino acid sequence of SEQ ID NO: 12; and/or a CDR3 of the single domain variable region (CDR3) comprising, consisting of, or consisting essentially of the amino acid sequence SEQ ID NO: 17.

In some embodiments, the anti-SARS-CoV-2 single domain antibody or single domain antibody fragment comprises: a CDR1 of the single domain variable region (CDR1) comprising, consisting of, or consisting essentially of the amino acid sequence SEQ ID NO: 7; a CDR2 of the single domain variable region (CDR2) comprising, consisting of, or consisting essentially of the amino acid sequence of SEQ ID NO: 13; and/or a CDR3 of the single domain variable region (CDR3) comprising, consisting of, or consisting essentially of the amino acid sequence SEQ ID NO: 18.

In some embodiments, the anti-SARS-CoV-2 single domain antibody or single domain antibody fragment comprises, consists of, or consists essentially of a CDR3 of the single domain variable region (CDR3) comprising, consisting of, or consisting essentially of the amino acid sequence of (a) SEQ ID NO: 17; (b) SEQ ID NO: 18; (c) SEQ ID NO: 19; or (d) SEQ ID NO: 20, wherein the single domain antibody or single domain antibody fragment does not comprise either an amino acid sequence of a CDR1 of the single domain variable region (CDR1) or a CDR2 of the single domain variable region (CDR2).

In some embodiments, the anti-SARS-CoV-2 single domain antibody or single domain antibody fragment comprises: a CDR1 of the single domain variable region (CDR1) comprising, consisting of, or consisting essentially of the amino acid sequence of (a) SEQ ID NO: 8; (b) SEQ ID NO: 9; or (c) SEQ ID NO: 10; a CDR2 of the single domain variable region (CDR2) comprising, consisting of, or consisting essentially of the amino acid sequence of (a) SEQ ID NO: 14; (b) SEQ ID NO: 15; or (c) SEQ ID NO: 16; and/or a CDR3 of the single domain variable region (CDR3) comprising, consisting of, or consisting essentially of the amino acid sequence of (a) SEQ ID NO: 19; or (b) SEQ ID NO: 20.

In some embodiments, the anti-SARS-CoV-2 single domain antibody or single domain antibody fragment comprises: a CDR1 of the single domain variable region (CDR1) comprising, consisting of, or consisting essentially of the amino acid sequence of SEQ ID NO: 8; a CDR2 of the single domain variable region (CDR2) comprising, consisting of, or consisting essentially of the amino acid sequence of SEQ ID NO: 14; and/or a CDR3 of the single domain variable region (CDR3) comprising, consisting of, or consisting essentially of the amino acid sequence of SEQ ID NO: 19.

In some embodiments, the anti-SARS-CoV-2 single domain antibody or single domain antibody fragment comprises: a CDR1 of the single domain variable region (CDR1) comprising, consisting of, or consisting essentially of the amino acid sequence of SEQ ID NO: 9; a CDR2 of the single domain variable region (CDR2) comprising, consisting of, or consisting essentially of the amino acid sequence of SEQ ID NO: 15; and/or a CDR3 of the single domain variable region (CDR3) comprising, consisting of, or consisting essentially of the amino acid sequence of SEQ ID NO: 19.

In some embodiments, the anti-SARS-CoV-2 single domain antibody or single domain antibody fragment comprises: a CDR1 of the single domain variable region (CDR1) comprising, consisting of, or consisting essentially of the amino acid sequence of SEQ ID NO: 10; a CDR2 of the single domain variable region (CDR2) comprising, consisting of, or consisting essentially of the amino acid sequence of SEQ ID NO: 16; and/or a CDR3 of the single domain variable region (CDR3) comprising, consisting of, or consisting essentially of the amino acid sequence of SEQ ID NO: 20.

In some embodiments, the single domain antibody or single domain antibody fragment comprises an amino acid sequence that is at least 90% identical (e.g., at least 91%, at least 92%, at least 93%, at least 94%, at least 95%, at least 96%, at least 97%, at least 98%, at least 99%, or 100% identical) to any of the foregoing variable region sequences.

In one embodiment, the SARS-CoV-2 single domain antibody or single domain antibody fragment has an immunoglobulin single domain variable region comprising, consisting of, or consisting essentially of SEQ ID NO: 1 or SEQ ID NO: 2, or at least the CDR regions thereof, wherein the CDRs are as determined with determined in accordance with any of the various known immunoglobulin numbering schemes, particularly in accordance with Kabat, Chothia, Martin (Enhanced Chothia), IGMT, or AHo. For example, in some embodiments, the single domain antibody or single domain antibody fragment comprises a single domain variable region of SEQ ID NO: 1, or at least the CDRs thereof, as determined by Kabat. In some embodiments, the single domain antibody or single domain antibody fragment comprises a single domain variable region of SEQ ID NO: 1, or at least the CDRs thereof, as determined by Chothia. In some embodiments, the single domain antibody or single domain antibody fragment comprises a single domain variable region of SEQ ID NO: 1, or at least the CDRs thereof, as determined by Martin. In some embodiments, the single domain antibody or single domain antibody fragment comprises a single domain variable region of SEQ ID NO: 1, or at least the CDRs thereof, as determined by IGMT. In some embodiments, the single domain antibody or single domain antibody fragment comprises a single domain variable region of SEQ ID NO: 1, or at least the CDRs thereof, as determined by AHo.

In some embodiments, the single domain antibody or single domain antibody fragment comprises a single domain variable region of SEQ ID NO: 2, or at least the CDRs thereof, as determined by Kabat. In some embodiments, the single domain antibody or single domain antibody fragment comprises a single domain variable region of SEQ ID NO: 2, or at least the CDRs thereof, as determined by Chothia. In some embodiments, the single domain antibody or single domain antibody fragment comprises a single domain variable region of SEQ ID NO: 2, or at least the CDRs thereof, as determined by Martin. In some embodiments, the single domain antibody or single domain antibody fragment comprises a single domain variable region of SEQ ID NO: 2, or at least the CDRs thereof, as determined by IGMT. In some embodiments, the single domain antibody or single domain antibody fragment comprises a single domain variable region of SEQ ID NO: 2, or at least the CDRs thereof, as determined by AHo.

The SARS-CoV-2 binding agent (e.g., antibody or antibody fragment) can also be a binding agent that competes with (e.g., binds to the same epitope or an overlapping epitope as) a SARS-CoV-2 binding agent comprising a single domain variable region polypeptide described herein for binding to SARS-CoV-2 (e.g., a binding agent comprising a single domain comprising SEQ ID NO: 1 or SEQ ID NO: 2). Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

Furthermore, the anti-SARS-CoV-2 single domain antibody or single domain antibody fragment can comprise an immunoglobulin single domain variable region having a specified percent identity to the single domain variable region sequence, such as at least 90% identical (e.g., at least 91%, at least 92%, at least 93%, at least 94%, at least 95%, at least 96%, at least 97%, at least 98%, at least 99%, or 100% identical). In some embodiments, the variance in sequence occurs outside the CDRs (as determined by any known method including Kabat, Chothia, Martin (Enhanced Chothia), IGMT, or AHo), such that the single domain sequences having the specified sequence identity to the specific sequences set forth herein retain the CDRs of such sequences. In an embodiment, the SARS-CoV-2 single domain antibody or single domain antibody fragment comprises an immunoglobulin single domain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 1 or SEQ ID NO: 2, optionally wherein the sequence retains the CDRs of SEQ ID NO: 1 or SEQ ID NO: 2, wherein the CDRs are as determined in accordance with any of the various known immunoglobulin numbering schemes, particularly in accordance with Kabat, Chothia, Martin (Enhanced Chothia), IGMT, or AHo. In an embodiment, the SARS-CoV-2 single domain antibody or single domain antibody fragment comprises an immunoglobulin single domain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 1 or SEQ ID NO: 2, optionally wherein the sequence retains the CDRs of SEQ ID NO: 1 or SEQ ID NO: 2 with the exception of 1, 2, 3, 4, or 5 substitutions in the sequence of the CDRs.

Nucleic acid or amino acid sequence “identity,” as described herein, can be determined by comparing a nucleic acid or amino acid sequence of interest to a reference nucleic acid or amino acid sequence. The percent identity is the number of nucleotides or amino acid residues that are the same (i.e., that are identical) as between the sequence of interest and the reference sequence divided by the length of the longest sequence (i.e., the length of either the sequence of interest or the reference sequence, whichever is longer). A number of mathematical algorithms for obtaining the optimal alignment and calculating identity between two or more sequences are known and incorporated into a number of available software programs. Examples of such programs include CLUSTAL-W, T-Coffee, and ALIGN (for alignment of nucleic acid and amino acid sequences), BLAST programs (e.g., BLAST 2.1, BL2SEQ, and later versions thereof) and FASTA programs (e.g., FASTA3x, FAS™, and SSEARCH) (for sequence alignment and sequence similarity searches). Sequence alignment algorithms also are disclosed in, for example, Altschul et al., J. Molecular Biol., 215(3): 403-410 (1990), Beigert et al., Proc. Natl. Acad. Sci. USA, 106(10): 3770-3775 (2009), Durbin et al., eds., Biological Sequence Analysis: Probabalistic Models of Proteins and Nucleic Acids, Cambridge University Press, Cambridge, UK (2009), Soding, Bioinformatics, 21(7): 951-960 (2005), Altschul et al., Nucleic Acids Res., 25(17): 3389-3402 (1997), and Gusfield, Algorithms on Strings, Trees and Sequences, Cambridge University Press, Cambridge UK (1997)).

### II. Antibody or Antibody-Fragment

In some embodiments, the SARS-CoV-2 binding agent is an anti-SARS-CoV-2 antibody or antigen-binding antibody fragment. An antibody or antigen-binding antibody fragment, as the terms are used in the “Antibody or Antibody-fragment” section, comprises, consist of, or consists essentially of an immunoglobulin heavy chain polypeptide and an immunoglobulin light chain polypeptide, or at least a portion of one of the variable regions (e.g., antigen-binding fragments) thereof.

A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the β sheets that provide the structural framework of the variable region (see, e.g., C. A. Janeway et al. (eds.), Immunobiology, 5th Ed., Garland Publishing, New York, NY (2001)).

The framework regions are connected by three complementarity determining regions (CDRs). As discussed above, the three CDRs, known as CDR1, CDR2, and CDR3, form the “hypervariable region” of an antibody, which is responsible for antigen binding. The CDR regions also can be referred to using an “H” or “L” in the nomenclature to denote the heavy or light chain, respectively, i.e., CDRH1, CDRH2, CDRH3, CDRL1, CDRL2, or CDRL3.

In some embodiments, the immunoglobulin heavy chain variable region comprises, consists of, or consists essentially of the amino acid sequence of Gln Val Gln Leu Gln Glu Ser Gly Gly Gly Leu Val Gln Thr Gly Gly Ser Leu Arg Leu Ser Cys Ala Ala Ser Gly Ser Phe Phe Ser Ile Asn Asp Met Gly Trp Tyr Arg Gln Ala Pro Gly Lys Gln Arg Glu Leu Val Ala Met Ile Thr Gly Asp Asp Ser Thr Asn Tyr Ala Asp Ser Val Lys Gly Arg Phe Thr Ile Ser Arg Asp Asn Leu Lys Asn Thr Val Tyr Leu Gln Met Asn Ser Leu Lys Pro Glu Asp Thr Ala Leu Tyr Tyr Cys Asn Ala Glu Val Lys Val Ile Ile Trp Asp Ser Arg Phe Asn Tyr Tyr Trp Gly Gln Gly Thr Gln Val Thr Val Ser Ser (SEQ ID NO: 1), or at least the CDRs thereof.

In other embodiments, the immunoglobulin heavy chain variable region comprises, consists of, or consists essentially of the amino acid sequence of Xaa1 Val Xaa2 Leu Xaa3 Xaa4 Xaa5 Gly Gly Xaa6 Xaa7 Val Xaa8 Xaa9 Gly Xaa10 Ser Leu Xaa11 Leu Ser Cys Xaa12 Xaa13 Ser Xaa14 Xaa15 Xaa16 Xaa17 Xaa18 Xaa19 Xaa20 Xaa21 Xaa22 Xaa23 Trp Xaa24 Arg Gln Xaa25 Pro Gly Xaa26 Xaa27 Arg Glu Xaa28 Val Xaa29 Xaa30 Xaa31 Xaa32 Xaa33 Xaa34 Xaa35 Xaa36 Xaa37 Xaa38 Xaa39 Xaa40 Xaa41 Xaa42 Xaa43 Xaa44 Xaa45 Arg Xaa46 Xaa47 Xaa48 Ser Xaa49 Asp Xaa50 Xaa51 Xaa52 Xaa53 Xaa54 Xaa55 Xaa56 Leu Xaa57 Xaa58 Xaa59 Xaa60 Leu Xaa61 Xaa62 Xaa63 Asp Thr Ala Xaa64 Tyr Xaa65 Cys Xaa66 Xaa67 Xaa68 Xaa69 Xaa70 Xaa71 Xaa72 Xaa73 Xaa74 Xaa75 Xaa76 Xaa77 Xaa78 Xaa79 Xaa80 Xaa81 Trp Gly Xaa82 Gly Thr Xaa83 Val Thr Val Ser Ser (SEQ ID NO: 2), or at least the CDRs thereof, wherein (a) Xaa1 is glutamine (Gln) or glutamic acid (Glu), (b) Xaa2 is glutamine (Gln), glutamic acid (Glu), or Lysine (Lys), (c) Xaa3 is glutamine (Gln), valine (Val), or alanine (Ala), (d) Xaa4 is glutamine (Gln), valine (Val), or alanine (Ala), (e) Xaa5 is serine (Ser) or threonine (Thr), (f) Xaa6 is glycine (Gly) or aspartic acid (Asp), (g) Xaa7 is leucine (Leu), serine (Ser), or cysteine (Cys), (h) Xaa8 is glutamine (Gln), arginine (Arg), or glutamic acid (Glu), (i) Xaa9 is threonine (Thr), alanine (Ala), or proline (Pro), (j) Xaa10 is glycine (Gly), aspartic acid (Asp), or glutamic acid (Glu), (k) Xaa11 is arginine (Arg), threonine (Thr), or serine (Ser), (1) Xaa12 is alanine (Ala), valine (Val), threonine (Thr), or lysine (Lys), (m) Xaa13 is alanine (Ala), valine (Val), or threonine (Thr), (n) Xaa14 is glycine (Gly), alanine (Ala), valine (Val), or arginine (Arg), (o) Xaa15 is serine (Ser), arginine (Arg), threonine (Thr), leucine (Leu), or aspartic acid (Asp), (p) Xaa16 is phenylalanine (Phe), threonine (Thr), or isoleucine (Ile), (q) Xaa17 is phenylalanine (Phe), valine (Val), leucine (Leu), or serine (Ser), (r) Xaa18 is serine (Ser), arginine (Arg), glutamine (Gln), or asparagine (Asn), (s) Xaa19 is isoleucine (Ile), serine (Ser), threonine (Thr), phenylalanine (Phe), leucine (Leu), lysine (Lys), asparagine (Asn), glutamic acid (Glu), or valine (Val), (t) Xaa20 is asparagine (Asn), tyrosine (Tyr), isoleucine (Ile), phenylalanine (Phe), or glycine (Gly), (u) Xaa21 is aspartic acid (Asp), threonine (Thr), serine (Ser), alanine (Ala), glutamine (Gln), arginine (Arg), or valine (Val), (v) Xaa22 is methionine (Met), valine (Val), or isoleucine (Ile), (w) Xaa23 is glycine (Gly), arginine (Arg), threonine (Thr), tyrosine (Tyr), alanine (Ala), or asparagine (Asn), (x) Xaa24 is tyrosine (Tyr) or phenylalanine (Phe), (y) Xaa25 is alanine (Ala) or threonine (Thr), (z) Xaa26 is lysine (Lys), threonine (Thr), or serine (Ser), (aa) Xaa27 is glutamine (Gln) or glutamic acid (Glu), (ab) Xaa28 is leucine (Leu) or phenylalanine (Phe), (ac) Xaa29 is alanine (Ala) or serine (Ser), (ad) Xaa30 is methionine (Met), alanine (Ala), threonine (Thr), or serine (Ser), (ae) Xaa31 is isoleucine (Ile), serine (Ser), threonine (Thr), or arginine (Arg), (af) Xaa32 is threonine (Thr), valine (Val), glutamine (Gln), asparagine (Asn), or serine (Ser), (ag) Xaa33 is glycine (Gly), serine (Ser), aspartic acid (Asp), threonine (Thr), glycine (Gly), asparagine (Asn), alanine (Ala), or tryptophan (Trp), (ah) Xaa34 is aspartic acid (Asp), glycine (Gly), serine (Ser), or isoleucine (Ile), (ai) Xaa35 is aspartic acid (Asp), glycine (Gly), tyrosine (Tyr), or arginine (Arg), (aj) Xaa36 is serine (Ser), arginine (Arg), asparagine (Asn), or alanine (Ala), (ak) Xaa37 is threonine (Thr) or isoleucine (Ile), (al) Xaa38 is asparagine (Asn), tyrosine (Tyr), threonine (Thr), serine (Ser), lysine (Lys), arginine (Arg), aspartic acid (Asp), or histidine (His), (am) Xaa39 is tyrosine (Tyr) or histidine (His), (an) Xaa40 is alanine (Ala), glutamic acid (Glu), aspartic acid (Asp), valine (Val), isoleucine (Ile), threonine (Thr), serine (Ser), or lysine (Lys), (ao) Xaa41 is aspartic acid (Asp) or glutamic acid (Glu), (ap) Xaa42 is serine (Ser) or phenylalanine (Phe), (aq) Xaa43 is valine (Val) or alanine (Ala), (ar) Xaa44 is lysine (Lys), threonine (Thr), or glutamic acid (Glu), (as) Xaa45 is glycine (Gly) or aspartic acid (Asp), (at) Xaa46 is phenylalanine (Phe) or valine (Val), (au) Xaa47 is threonine (Thr) or alanine (Ala), (av) Xaa48 is isoleucine (Ile) or alanine (Ala), (aw) Xaa49 is arginine (Arg) or glycine (Gly), (ax) Xaa50 is asparagine (Asn), threonine (Thr), phenylalanine (Phe), or aspartic acid (Asp), (ay) Xaa51 is leucine (Leu), alanine (Ala), valine (Val), methionine (Met), serine (Ser), or aspartic acid (Asp), (az) Xaa52 is lysine (Lys), glutamine (Gln), or glutamic acid (Glu), (ba) Xaa53 is asparagine (Asn), threonine (Thr), glycine (Gly), histidine (His), or alanine (Ala), (bb) Xaa54 is threonine (Thr), leucine (Leu), or isoleucine (Ile), (bc) Xaa55 is valine (Val) or aspartic acid (Asp), (bd) Xaa56 is tyrosine (Tyr), serine (Ser), histidine (His), or aspartic acid (Asp), (be) Xaa57 is glutamine (Gln), aspartic acid (Asp), or glutamic acid (Glu), (bf) Xaa58 is methionine (Met), leucine (Leu), or valine (Val), (bg) Xaa59 is asparagine (Asn), serine (Ser), or threonine (Thr), (bh) Xaa60 is serine (Ser), aspartic acid (Asp), or asparagine (Asn), (bi) Xaa61 is lysine (Lys) or glutamic acid (Glu), (bj) Xaa62 is proline (Pro), valine (Val), or serine (Ser), (bk) Xaa63 is glutamic acid (Glu), lysine (Lys), or aspartic acid (Asp), (bl) Xaa64 is leucine (Leu) or valine (Val), (bm) Xaa65 is tyrosine (Tyr), arginine (Arg), or threonine (Thr), (bn) Xaa66 is asparagine (Asn), alanine (Ala), histidine (His), lysine (Lys), valine (Val), or threonine (Thr), (bo) Xaa67 is alanine (Ala), arginine (Arg), tyrosine (Tyr), proline (Pro), lysine (Lys), valine (Val), or threonine (Thr), (bp) Xaa68 is glutamic acid (Glu), aspartic acid (Asp), alanine (Ala), threonine (Thr), lysine (Lys), arginine (Arg), or serine (Ser), (bq) Xaa69 is valine (Val), arginine (Arg), glycine (Gly), lysine (Lys), proline (Pro), leucine (Leu), aspartic acid (Asp), or phenylalanine (Phe), (br) Xaa70 is lysine (Lys), glycine (Gly), tyrosine (Tyr), serine (Ser), arginine (Arg), aspartic acid (Asp), or glutamic acid (Glu), (bs) Xaa71 is valine (Val), tyrosine (Tyr), phenylalanine (Phe), tryptophan (Trp), glycine (Gly), isoleucine (Ile), or proline (Pro), (bt) Xaa72 is isoleucine (Ile), valine (Val), tyrosine (Tyr), phenylalanine (Phe), serine (Ser), glutamine (Gln), or arginine (Arg), (bu) Xaa73 is isoleucine (Ile), valine (Val), leucine (Leu), serine (Ser), threonine (Thr), tyrosine (Tyr), or cysteine (Cys), (by) Xaa74 is tryptophan (Trp), tyrosine (Tyr), phenylalanine (Phe), leucine (Leu), valine (Val), threonine (Thr), or proline (Pro), (bw) Xaa75 is aspartic acid (Asp), arginine (Arg), alanine (Ala), proline (Pro), serine (Ser), arginine (Arg), proline (Pro), or cysteine (Cys), (bx) Xaa76 is serine (Ser), alanine (Ala), threonine (Thr), proline (Pro), cysteine (Cys), glutamine (Gln), aspartic acid (Asp), or tryptophan (Trp), (by) Xaa77 is arginine (Arg), lysine (Lys), aspartic acid (Asp), glutamic acid (Glu), threonine (Thr), proline (Pro), or tyrosine (Tyr), (bz) Xaa78 is phenylalanine (Phe), aspartic acid (Asp), tryptophan (Trp), lysine (Lys), tyrosine (Tyr), or serine (Ser), (ca) Xaa79 is asparagine (Asn), serine (Ser), aspartic acid (Asp), glutamic acid (Glu), phenylalanine (Phe), or tyrosine (Tyr), (cb) Xaa80 is tyrosine (Tyr), aspartic acid (Asp), glutamic acid (Glu), asparagine (Asn), alanine (Ala), or glycine (Gly), (cc) Xaa81 is tyrosine (Tyr), glutamic acid (Glu), valine (Val), serine (Ser), or threonine (Thr), (cd) Xaa82 is glutamine (Gln) or lysine (Lys), (ce) Xaa83 is glutamine (Gln), glutamic acid (Glu), arginine (Arg), leucine (Leu), or proline (Pro).

In some embodiments, the immunoglobulin heavy chain variable region comprises, consists of, or consists essentially of the amino acid sequence of SEQ ID NO: 2, wherein the amino acid sequence differs from the amino acid sequence of SEQ ID NO: 1 at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 79 or 80 residues, preferably at 1, 2, 3, 4, 5, 6, 7, 8, 9, or 10 residues, more preferably 1, 2, or 3 residues, even more preferably at 1 or 2 residues, and yet even more preferably at 1 residue.

In some embodiments, immunoglobulin heavy chain variable region comprises, consists of, or consists essentially of the amino acid sequence of SEQ ID NO: 1, except that the amino acid sequence differs from the amino acid sequence of SEQ ID NO: 1 at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 78, 79 or 80 residues, preferably at only 1, 2, 3, 4, 5, 6, 7, 8, 9, or 10 residues, more preferably at only 1, 2, or 3 residues, even more preferably at only 1 or 2 residues, yet even more preferably at only 1 residue.

In an embodiment, the immunoglobulin heavy chain polypeptide comprises, consists of, or consists essentially of the amino acid sequence of SEQ ID NO: 1 or SEQ ID NO: 2, or at least the CDRs thereof. In another embodiment, the immunoglobulin heavy chain polypeptide comprises, consists of, or consists essentially of the amino acid sequence of SEQ ID NO: 2, or at least the CDRs thereof, with any one of the aforementioned amino acid substitutions in any suitable combination.

In some embodiments, the anti-SARS-CoV-2 antibody or antibody fragment comprises: a CDR1 of the heavy chain variable region (HCDR1) comprising, consisting of, or consisting essentially of the amino acid sequence of (a) Gly Ser Phe Phe Ser Ile Asn (SEQ ID NO: 5); (b) Ile Asn Asp Met Gly (SEQ ID NO: 6); or (c) Gly Ser Phe Phe Ser Ile Asn Asp (SEQ ID NO: 7); a CDR2 of the heavy chain variable region (HCDR2) comprising, consisting of, or consisting essentially of the amino acid sequence of (a) Thr Gly Asp Asp Ser (SEQ ID NO: 11); (b) Met Ile Thr Gly Asp Asp Ser Thr Asn Tyr Ala Asp Ser Val Lys Gly (SEQ ID NO: 12); or (c) Ile Thr Gly Asp Asp Ser Thr (SEQ ID NO: 13); and/or a CDR3 of the heavy chain variable region (HCDR3) comprising, consisting of, or consisting essentially of the amino acid sequence of (a) Glu Val Lys Val Ile Ile Trp Asp Ser Arg Phe Asn Tyr Tyr (SEQ ID NO: 17); or (b) Asn Ala Glu Val Lys Val Ile Ile Trp Asp Ser Arg Phe Asn Tyr Tyr (SEQ ID NO: 18).

In another embodiment, the anti-SARS-CoV-2 antibody or antibody fragment comprises: a CDR1 of the heavy chain variable region (HCDR1) comprising, consisting of, or consisting essentially of the amino acid sequence of Gly Ser Phe Phe Ser Ile Asn (SEQ ID NO: 5); a CDR2 of the heavy chain variable region (HCDR2) comprising, consisting of, or consisting essentially of the amino acid sequence of Thr Gly Asp Asp Ser (SEQ ID NO: 11); and/or a CDR3 of the heavy chain variable region (HCDR3) comprising, consisting of, or consisting essentially of the amino acid sequence of Glu Val Lys Val Ile Ile Trp Asp Ser Arg Phe Asn Tyr Tyr (SEQ ID NO: 17).

In some embodiments, the anti-SARS-CoV-2 antibody or antibody fragment comprises: a CDR1 of the heavy chain variable region (HCDR1) comprising, consisting of, or consisting essentially of the amino acid sequence Ile Asn Asp Met Gly (SEQ ID NO: 6); a CDR2 of the heavy chain variable region (HCDR2) comprising, consisting of, or consisting essentially of the amino acid sequence of Met Ile Thr Gly Asp Asp Ser Thr Asn Tyr Ala Asp Ser Val Lys Gly (SEQ ID NO: 12); and/or a CDR3 of the heavy chain variable region (HCDR3) comprising, consisting of, or consisting essentially of the amino acid sequence of Glu Val Lys Val Ile Ile Trp Asp Ser Arg Phe Asn Tyr Tyr (SEQ ID NO: 17).

In some embodiments, the anti-SARS-CoV-2 antibody or antibody fragment comprises: a CDR1 of the heavy chain variable region (HCDR1) comprising, consisting of, or consisting essentially of the amino acid sequence of Gly Ser Phe Phe Ser Ile Asn Asp (SEQ ID NO: 7); a CDR2 of the heavy chain variable region (HCDR2) comprising, consisting of, or consisting essentially of the amino acid sequence of Ile Thr Gly Asp Asp Ser Thr (SEQ ID NO: 13); and/or a CDR3 of the heavy chain variable region (HCDR3) comprising, consisting of, or consisting essentially of the amino acid sequence of Asn Ala Glu Val Lys Val Ile Ile Trp Asp Ser Arg Phe Asn Tyr Tyr (SEQ ID NO: 18).

In some embodiments, the anti-SARS-CoV-2 antibody or antibody fragment comprises
a CDR1 of the heavy chain variable region (HCDR1) comprising, consisting of, or consisting essentially of the amino acid sequence of (a) Xaa1 Xaa2 Xaa3 Xaa4 Xaa5 Xaa6 Xaa7 (SEQ ID NO: 8); wherein Xaa1 is glycine (G), alanine (A), valine (V), or arginine (R); Xaa2 is serine (S), arginine (R), threonine (T), leucine (L), or aspartic acid (D); Xaa3 is phenylalanine (F), threonine (T), or isoleucine (I); Xaa4 is phenylalanine (F), valine (V), leucine (L), or serine (S); Xaa5 is serine (S), arginine (R), glutamine (Q), or asparagine (N); Xaa6 is isoleucine (I), serine (S), threonine (T), phenylalanine (F), leucine (L), lysine (K), asparagine (N), glutamic acid (E), or valine (V); and Xaa7 is asparagine (N), tyrosine (Y), isoleucine (I), phenylalanine (F), or glycine (G); (b) Xaa1 Xaa2 Xaa3 Xaa4 Xaa5 (SEQ ID NO: 9), wherein Xaa1 is isoleucine (I), serine (S), threonine (T), phenylalanine (F), leucine (L), lysine (K), asparagine (N), glutamic acid (E), or valine (V); Xaa2 is asparagine (N), tyrosine (Y), isoleucine (I), phenylalanine (F), or glycine (G); Xaa3 is aspartic acid (D), threonine (T), serine (S), alanine (A), glutamine (Q), arginine (R), or valine (V); Xaa4 is methionine (M), valine (V), or isoleucine (I), and Xaa5 is glycine (G), arginine (R), threonine (T), tyrosine (Y), alanine (A), or asparagine (N); or (c) Xaa1 Xaa2 Xaa3 Xaa4 Xaa5 Xaa6 Xaa7 Xaa8 (SEQ ID NO: 10); wherein Xaa1 is glycine (G), alanine (A), valine (V), or arginine (R); Xaa2 is serine (S), arginine (R), threonine (T), leucine (L), or aspartic acid (D); Xaa3 is phenylalanine (F), threonine (T), or isoleucine (I); Xaa4 is phenylalanine (F), valine (V), leucine (L), or serine (S); Xaa5 is serine (S), arginine (R), glutamine (Q), or asparagine (N); Xaa6 is isoleucine (I), serine (S), threonine (T), phenylalanine (F), leucine (L), lysine (K), asparagine (N), glutamic acid (E), or valine (V); Xaa7 is asparagine (N), tyrosine (Y), isoleucine (I), phenylalanine (F), or glycine (G); and Xaa8 is is aspartic acid (D), threonine (T), serine (S), alanine (A), glutamine (Q), arginine (R), or valine (V);
a CDR2 of the heavy chain variable region (HCDR2) comprising, consisting of, or consisting essentially of the amino acid sequence of (a) Xaa1 Xaa2 Xaa3 Xaa4 Xaa5 (SEQ ID NO: 14), wherein Xaa1 is threonine (T), valine (V), glutamine (Q), asparagine (N), or serine (S); Xaa2 is glycine (G), serine (S), aspartic acid (D), threonine (T), asparagine (N), alanine (A), or tryptophan (W); Xaa3 is aspartic acid (D), glycine (G), serine (S), or isoleucine (I); Xaa4 is aspartic acid (D), glycine (G), tyrosine (Y), or arginine (R); and Xaa5 is serine (S), arginine (R), asparagine (N), or alanine (A); (b) Xaa1 Xaa2 Xaa3 Xaa4 Xaa5 Xaa6 Xaa7 Xaa8 Xaa9 Xaa10 Xaa11 Xaa12 Xaa13 Xaa14 Xaa15 Xaa16 (SEQ ID NO: 15), wherein Xaa1 is methionine (M), alanine (A), threonine (T), or serine (S); Xaa2 is isoleucine (I), serine (S), threonine (T), or arginine (R); Xaa3 is threonine (T), valine (V), glutamine (Q), asparagine (N), or serine (S); Xaa4 is glycine (G), serine (S), aspartic acid (D), threonine (T), asparagine (N), alanine (A), or tryptophan (W); Xaa5 is aspartic acid (D), glycine (G), serine (S), or isoleucine (I); Xaa6 is aspartic acid (D), glycine (G), tyrosine (Y), or arginine (R); Xaa7 is serine (S), arginine (R), asparagine (N), or alanine (A); Xaa8 is threonine (T) or isoleucine (I); Xaa9 is asparagine (N), tyrosine (Y), threonine (T), serine (S), lysine (K), arginine (R), aspartic acid (D), or histidine (H); Xaa10 is tyrosine (Y) or histidine (H); Xaa11 is alanine (A), glutamic acid (E), aspartic acid (D), valine (V), isoleucine (I), threonine (T), serine (S), or lysine (K); Xaa12 is aspartic acid (D) or glutamic acid (E); Xaa13 is serine (S) or phenylalanine (F); Xaa14 is valine (V) or alanine (A); Xaa15 is lysine (K), threonine (T), or glutamic acid (E); and Xaa16 is glycine (G) or aspartic acid (D); or (c) Xaa1 Xaa2 Xaa3 Xaa4 Xaa5 Xaa6 Xaa7 (SEQ ID NO: 16); wherein Xaa1 is isoleucine (I), serine (S), threonine (T), or arginine (R); Xaa2 is threonine (T), valine (V), glutamine (Q), asparagine (N), or serine (S); Xaa3 is glycine (G), serine (S), aspartic acid (D), threonine (T), asparagine (N), alanine (A), or tryptophan (W); Xaa4 is aspartic acid (D), glycine (G), serine (S), or isoleucine (I); Xaa5 is aspartic acid (D), glycine (G), tyrosine (Y), or arginine (R); Xaa6 is serine (S), arginine (R), asparagine (N), or alanine (A); and Xaa7 is threonine (T) or isoleucine (I); and/or
a CDR3 of the heavy chain variable region (HCDR3) comprising, consisting of, or consisting essentially of the amino acid sequence of (a) Xaa1 Xaa2 Xaa3 Xaa4 Xaa5 Xaa6 Xaa7 Xaa8 Xaa9 Xaa10 Xaa11 Xaa12 Xaa13 Xaa14 (SEQ ID NO: 19); wherein Xaa1 is glutamic acid (E), aspartic acid (D), alanine (A), threonine (T), lysine (K), arginine (R), or serine (S); Xaa2 is valine (V), arginine (R), glycine (G), lysine (K), proline (P), leucine (L), aspartic acid (D), or phenylalanine (F); Xaa3 is lysine (K), glycine (G), tyrosine (Y), serine (S), arginine (R), aspartic acid (D), or glutamic acid (E); Xaa4 is valine (V), tyrosine (Y), phenylalanine (F), tryptophan (W), glycine (G), isoleucine (I), or proline (P); Xaa5 is isoleucine (I), valine (V), tyrosine (Y), phenylalanine (F), serine (S), glutamine (Q), or arginine (R); Xaa6 is isoleucine (I), valine (V), leucine (L), serine (S), threonine (T), tyrosine (Y), or cysteine (C); Xaa7 is tryptophan (W), tyrosine (Y), phenylalanine (F), leucine (L), valine (V), threonine (T), or proline (P); Xaa8 is aspartic acid (D), aspartic acid (D), arginine (R), alanine (A), proline (P), serine (S), arginine (R), proline (P), or cysteine (C); Xaa9 is serine (S), alanine (A), threonine (T), proline (P), cysteine (C), glutamine (Q), aspartic acid (D), or tryptophan (W); Xaa10 is arginine (R), lysine (K), aspartic acid (D), glutamic acid (E), threonine (T), proline (P), tyrosine (Y), or arginine (R); Xaa11 is phenylalanine (F), aspartic acid (D), tryptophan (W), lysine (K), tyrosine (Y), serine (S), or phenylalanine (F); Xaa12 is asparagine (N), serine (S), aspartic acid (D), glutamic acid (E), phenylalanine (F), or tyrosine (Y); Xaa13 is tyrosine (Y), aspartic acid (D), glutamic acid (E), asparagine (N), alanine (A), or glycine (G); and Xaa14 is tyrosine (Y), glutamic acid (E), valine (V), serine (S), or threonine (T); or (b) Xaa1 Xaa2 Xaa3 Xaa4 Xaa5 Xaa6 Xaa7 Xaa8 Xaa9 Xaa10 Xaa11 Xaa12 Xaa13 Xaa14 Xaa15 Xaa16 (SEQ ID NO: 20); wherein Xaa1 is asparagine (N), alanine (A), histidine (H), lysine (K), valine (V), or threonine (T); Xaa2 is alanine (A), arginine (R), tyrosine (Y), proline (P), lysine (K), valine (V), or threonine (T); Xaa3 is glutamic acid (E), aspartic acid (D), alanine (A), threonine (T), lysine (K), arginine (R), or serine (S); Xaa4 is valine (V), arginine (R), glycine (G), lysine (K), proline (P), leucine (L), aspartic acid (D), or phenylalanine (F); Xaa5 is lysine (K), glycine (G), tyrosine (Y), serine (S), arginine (R), aspartic acid (D), or glutamic acid (E); Xaa6 is valine (V), tyrosine (Y), phenylalanine (F), tryptophan (W), glycine (G), valine (V), isoleucine (I), or proline (P); Xaa7 is isoleucine (I), valine (V), tyrosine (Y), phenylalanine (F), serine (S), glutamine (Q), or arginine (R); Xaa8 is isoleucine (I), valine (V), leucine (L), serine (S), threonine (T), tyrosine (Y), or cysteine (C); Xaa9 is tryptophan (W), tyrosine (Y), phenylalanine (F), leucine (L), valine (V), threonine (T), or proline (P); Xaa10 is aspartic acid (D), arginine (R), alanine (A), proline (P), serine (S), arginine (R), proline (P), or cysteine (C); Xaa11 is serine (S), alanine (A), threonine (T), proline (P), cysteine (C), glutamine (Q), aspartic acid (D), or tryptophan (W); Xaa12 is arginine (R), lysine (K), aspartic acid (D), glutamic acid (E), threonine (T), proline (P), or tyrosine (Y); Xaa13 is phenylalanine (F), aspartic acid (D), tryptophan (W), lysine (K), tyrosine (Y), serine (S), or phenylalanine (F); Xaa14 is asparagine (N), serine (S), aspartic acid (D), glutamic acid (E), phenylalanine (F), or tyrosine (Y); Xaa15 is tyrosine (Y), aspartic acid (D), glutamic acid (E), asparagine (N), alanine (A), or glycine (G); and Xaa16 is tyrosine (Y), glutamic acid (E), valine (V), serine (S), or threonine (T).

In some embodiments, the anti-SARS-CoV-2 antibody or antibody fragment comprises: a CDR1 of the heavy chain variable region (HCDR1) comprising, consisting of, or consisting essentially of the amino acid sequence of SEQ ID NO: 8; a CDR2 of the heavy chain variable region (HCDR2) comprising, consisting of, or consisting essentially of the amino acid sequence of SEQ ID NO: 14; and/or a CDR3 of the heavy chain variable region (HCDR3) comprising, consisting of, or consisting essentially of the amino acid sequence of SEQ ID NO: 19.

In some embodiments, the anti-SARS-CoV-2 antibody or antibody fragment comprises: a CDR1 of the heavy chain variable region (HCDR1) comprising, consisting of, or consisting essentially of the amino acid sequence of SEQ ID NO: 9; a CDR2 of the heavy chain variable region (HCDR2) comprising, consisting of, or consisting essentially of the amino acid sequence of SEQ ID NO: 15; and/or a CDR3 of the heavy chain variable region (HCDR3) comprising, consisting of, or consisting essentially of the amino acid sequence of SEQ ID NO: 19.

In some embodiments, the anti-SARS-CoV-2 antibody or antibody fragment comprises: a CDR1 of the heavy chain variable region (HCDR1) comprising, consisting of, or consisting essentially of the amino acid sequence of SEQ ID NO: 10; a CDR2 of the heavy chain variable region (HCDR2) comprising, consisting of, or consisting essentially of the amino acid sequence of SEQ ID NO: 16; and/or a CDR3 of the heavy chain variable region (HCDR3) comprising, consisting of, or consisting essentially of the amino acid sequence of SEQ ID NO: 20.

In some embodiments, the immunoglobulin heavy chain polypeptide comprises an amino acid sequence that is at least 90% identical (e.g., at least 91%, at least 92%, at least 93%, at least 94%, at least 95%, at least 96%, at least 97%, at least 98%, at least 99%, or 100% identical) to any of the foregoing variable region sequences.

In addition to an immunoglobulin heavy chain polypeptide as described herein, the anti-SARS-CoV-2 antibody or antibody fragment comprises an immunoglobulin light chain polypeptide that comprises, consists of, or consists essentially of a variable region that comprises, consists of, or consists essentially of any suitable amino acid sequence that allows for specific binding of the antibody or antigen-binding antibody fragment to an epitope of SARS-CoV-2. As described above, the anti-SARS-CoV-2 antibody or antibody fragment can comprise an immunoglobulin heavy chain variable region and light chain variable region have any of the foregoing heavy and light chain variable region sequences, or the CDRs thereof. The CDR sequences can be the CDR sequences set forth herein or the CDR sequences as determined using any of several known methods (e.g., Kabat, Chothia, Martin (Enhanced Chothia), IGMT, or AHo).

In some embodiments, the anti-SARS-CoV-2 antibody or antibody fragment binds an epitope of the RBD protein of SARS-CoV-2 comprising, consisting of, or consisting essentially of, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 amino acid residues of the amino acid sequence of SEQ ID NO: 21.

SEQ ID NO: 21 is a significant portion of the Wuhan SARS-CoV-2 strain Spike protein RBD, the sequence of which is as follows: NITNLCPFGEVFNATRFASVYAWNRKRISNCVADYSVLYNSASFSTFKCYGVSPTKLND LCFTNVYADSFVIRGDEVRQIAPGQTGKIADYNYKLPDDFTGCVIAWNSNNLDSKVGGN YNYLYRLFRKSNLKPFERDISTEIYQAGSTPCNGVEGFNCYFPLQSYGFQPTNGVGYQPY RVVVLSFELLHAPATVCGPKKSTNLVKNK. Although sources vary on the starting points and end points of the RBD region within the SARS-CoV-2 Spike protein, the sequence of SEQ ID NO: 21 falls within the RBD region of known starting points and end points.

In some embodiments, the epitope comprises, consists of, or consists essentially of 5, 6, 7, 8, 9, 10, 11, 12, 13, 14, 15, 16, 17, 18, 19, 20, 21, 22, 23, 24 or 25 amino acid residues. In one embodiment, the epitope comprises, consists of, or consists essentially of the amino acid residues at positions 45-48, 50, 74, 75, 77-81, 84, 103, 170-175 and 178 relative to SEQ ID NO: 21. In one embodiment, the epitope comprises, consists of, or consists essentially of the amino acid residues at positions 45, 47, 50, 77, 80, 103, 171, 173, and 178 relative to SEQ ID NO: 21. In one embodiment, the epitope comprises, consists of, or consists essentially of the amino acid residues at positions 47, 50, 77, 80, 103, 173, and 178 relative to SEQ ID NO: 21. In one embodiment, the epitope comprises, consists of, or consists essentially of the amino acid residues F47, K48, Y50, G74, V77, Q79, I80, A81, Q84, V103, T170, G172, V173, G174, Y178, and optionally, at least one of S45, F45, T46, A46, D75, N75, R78, S78, T171, N171, Y171, I173, Y175, H175, wherein the residue numbering is relative to SEQ ID NO: 21. In another embodiment, the epitope comprises, consists of, or consists essentially of amino acid residues F47, K48, Y50, G74, V77, Q79, I80, A81, Q84, V103, T170, G172, V173, G174, and Y178, wherein the residue numbering is relative to SEQ ID NO: 21. In another embodiment, the epitope comprises, consists of, or consists essentially of amino acid residues F47, K48, Y50, G74, V77, Q79, I80, A81, Q84, V103, T170, G172, V173, G174, and Y178, wherein the residue numbering is relative to SEQ ID NO: 21. In another embodiment, the epitope comprises, consists of, or consists essentially of amino acid residues F45, F47, Y50, V77, I80, V103, Y171, V173, and Y178, wherein the residue numbering is relative to SEQ ID NO: 21. In another embodiment, the epitope comprises, consists of, or consists essentially of amino acid residues F47, Y50, V77, I80, V103, V173, and Y178, wherein the residue numbering is relative to SEQ ID NO: 21. In certain embodiments, the epitope comprises, consists of, or consists essentially of discontinuous segments of amino acid residues of SEQ ID NO: 21, wherein each segment comprises, consists of, or consists essentially of at least one amino acid residue. In certain embodiments, the epitope comprises, consists of, or consists essentially of a continuous sequence of amino acid residues comprising SEQ ID NO: 21 or a portion thereof. In certain embodiments, the anti-SARS-CoV-2 antibody or antibody fragment binds an epitope of the RBD protein of SARS-CoV-2, wherein after binding, the interaction or binding between the RBD protein and its target, e.g., an ACE2 receptor is reduced or inhibited.

In some embodiments, a polypeptide comprises, consists of, or consists essentially of, the epitiope. The polypeptide can be bound to a support, directly or via a linker molecule. The support can be any type of support (e.g., solid supports, such as a bead or plate), particularly a support useful in biopanning techniques, such as panning a phage display library.

The polypeptide can be used for any suitable purpose. For instance, the polypeptide can be used to screen for, select, or produce anti-SARS-CoV-2 antibodies. Thus, provided herein is a method of screening, selecting, or producing anti-SARS-CoV-2 antibodies by contacting one or more antibodies or antibody fragments (e.g., a library, such as a phage display library) with the polypeptide and selecting an antibody or antibody fragment that binds to the polypeptide. In some embodiments, the method comprises repeatedly performing the contacting and selection steps (e.g., panning) using the polypeptide and selecting those antibodies or antibody fragments that exhibit the greatest affinity for the polypeptide. Specific techniques for panning antibody libraries using polypeptides are known in the art. For instance, the polypeptide can be used in conjunction with panning phage display libraries.

In one embodiment, the SARS-CoV-2 antibody or antibody fragment has an immunoglobulin heavy chain variable region comprising, consisting of, or consisting essentially of SEQ ID NO: 1 or SEQ ID NO: 2, or at least the CDR regions thereof; and an immunoglobulin light chain variable region, or at least the CDR sequences thereof, wherein the CDRs are as determined with determined in accordance with any of the various known immunoglobulin numbering schemes, particularly in accordance with Kabat, Chothia, Martin (Enhanced Chothia), IGMT, or AHo. For example, in some embodiments, the antibody or antibody fragment comprises a heavy chain variable region of SEQ ID NO: 1, or at least the CDRs thereof, and a light chain variable region, or at least the CDRs thereof, as determined by Kabat. In some embodiments, the antibody or antibody fragment comprises a heavy chain variable region of SEQ ID NO: 1, or at least the CDRs thereof, and a light chain variable region, or at least the CDRs thereof, as determined by Chothia. In some embodiments, the antibody or antibody fragment comprises a heavy chain variable region of SEQ ID NO: 1, or at least the CDRs thereof, and a light chain variable region, or at least the CDRs thereof, as determined by Martin. In some embodiments, the antibody or antibody fragment comprises a heavy chain variable region of SEQ ID NO: 1, or at least the CDRs thereof, and a light chain variable region, or at least the CDRs thereof, as determined by IGMT. In some embodiments, the antibody or antibody fragment comprises a heavy chain variable region of SEQ ID NO: 1, or at least the CDRs thereof, and a light chain variable region, or at least the CDRs thereof, as determined by AHo.

In some embodiments, the antibody or antibody fragment comprises a heavy chain variable region of SEQ ID NO: 2, or at least the CDRs thereof, and a light chain variable region, or at least the CDRs thereof, as determined by Kabat. In some embodiments, the antibody or antibody fragment comprises a heavy chain variable region of SEQ ID NO: 2, or at least the CDRs thereof, and a light chain variable region, or at least the CDRs thereof, as determined by Chothia. In some embodiments, the antibody or antibody fragment comprises a heavy chain variable region of SEQ ID NO: 2, or at least the CDRs thereof, and a light chain variable region, or at least the CDRs thereof, as determined by Martin. In some embodiments, the antibody or antibody fragment comprises a heavy chain variable region of SEQ ID NO: 2, or at least the CDRs thereof, and a light chain variable region, or at least the CDRs thereof, as determined by IGMT. In some embodiments, the antibody or antibody fragment comprises a heavy chain variable region of SEQ ID NO: 2, or at least the CDRs thereof, and a light chain variable region, or at least the CDRs thereof, as determined by AHo.

Furthermore, the anti-SARS-CoV-2 antibody or antibody fragment can comprise an immunoglobulin heavy chain variable region and light chain variable region having specified percent identities to the heavy and light chain variable region sequences, such as at least 90% identical (e.g., at least 91%, at least 92%, at least 93%, at least 94%, at least 95%, at least 96%, at least 97%, at least 98%, at least 99%, or 100% identical). In some embodiments, the variance in sequence occurs outside the CDRs (as determined by any known method including Kabat, Chothia, Martin (Enhanced Chothia), IGMT, or AHo), such that the heavy and light chain sequences having the specified sequence identity to the specific sequences set forth herein retain the CDRs of such sequences. In an embodiment, the SARS-CoV-2 antibody or antibody fragment comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 1 or SEQ ID NO: 2, optionally wherein the sequence retains the CDRs of SEQ ID NO: 1 or SEQ ID NO: 2, wherein the CDRs are as determined in accordance with any of the various known immunoglobulin numbering schemes, particularly in accordance with Kabat, Chothia, Martin (Enhanced Chothia), IGMT, or AHo. In an embodiment, the SARS-CoV-2 antibody or antibody fragment comprises an immunoglobulin heavy chain variable region that is at least 90% identical (e.g., at least 91%, at least 92%, at least 93%, at least 94%, at least 95%, at least 96%, at least 97%, at least 98%, at least 99%, or 100% identical) to SEQ ID NO: 1 or SEQ ID NO: 2, optionally wherein the sequence retains the CDRs of SEQ ID NO: 1 or SEQ ID NO: 2 with the exception of 1, 2, 3, 4, or 5 substitutions in the sequence of the CDRs.

Variation in sequence identity can be accomplished through addition, substitution, or deletion of one or more amino acid residues. An amino acid “replacement” or “substitution” refers to the replacement of one amino acid at a given position or residue by another amino acid at the same position or residue within a polypeptide sequence. The amino acid replacement or substitution can be conservative, semi-conservative, or non-conservative depending upon whether the substitution is by an amino acid residue that has similar properties to the residue being replaced. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra).

Amino acids can be broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or Ile), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gln), lysine (K or Lys), and arginine (R or Arg).

Aliphatic amino acids may be sub-divided into four sub-groups. The “large aliphatic non-polar sub-group” consists of valine, leucine, and isoleucine. The “aliphatic slightly-polar sub-group” consists of methionine, serine, threonine, and cysteine. The “aliphatic polar/charged sub-group” consists of glutamic acid, aspartic acid, asparagine, glutamine, lysine, and arginine. The “small-residue sub-group” consists of glycine and alanine. The group of charged/polar amino acids may be sub-divided into three sub-groups: the “positively-charged sub-group” consisting of lysine and arginine, the “negatively-charged sub-group” consisting of glutamic acid and aspartic acid, and the “polar sub-group” consisting of asparagine and glutamine.

Aromatic amino acids may be sub-divided into two sub-groups: the “nitrogen ring sub-group” consisting of histidine and tryptophan and the “phenyl sub-group” consisting of phenylalanine and tyrosine.

Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free —OH can be maintained, and glutamine for asparagine such that a free —NH₂ can be maintained. “Semi-conservative mutations” include amino acid substitutions of amino acids within the same groups listed herein, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

When the immunoglobulin light chain or heavy chain variable region “consists essentially of” any of the foregoing heavy or light chain variable region amino acid sequences, additional components can be included in the polypeptide that do not materially affect the polypeptide, such as those described herein. When the immunoglobulin light chain or heavy chain variable region “consists of”, the polypeptide does not comprise any additional components.

The SARS-CoV-2 antibody or antibody fragment can be a binding agent that competes with a SARS-CoV-2 binding agent comprising an immunoglobulin heavy chain polypeptide and/or light chain polypeptide described herein for binding to SARS-CoV-2, e.g., binds to the same epitope or an overlapping epitope. Antibody competition can be assayed using routine peptide competition assays which utilize ELISA, Western blot, or immunohistochemistry methods (see, e.g., U.S. Pat. Nos. 4,828,981 and 8,568,992; and Braitbard et al., Proteome Sci., 4: 12 (2006)).

The SARS-CoV-2 binding agent of the present inventive method can be a whole antibody, or an antibody fragment, or one or more CDRs as determined by any known method (e.g., Kabat), or fragment of a CDR that retains the specific binding capacity of the full CDR, as described herein. The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The SARS-CoV-2 binding agent can contain any SARS-CoV-2 binding antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)₂ fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), (vi) a single chain Fv (scFv), which is a monovalent molecule consisting of the two domains of the Fv fragment (i.e., V_{L} and V_{H}) joined by a synthetic linker which enables the two domains to be synthesized as a single polypeptide chain, and (vii) a domain antibody (dAb), which is an antibody single variable region domain (VH or VL) polypeptide that specifically binds antigen.

When the SARS-CoV-2 binding agent is an antibody or antibody fragment, the antibody or antibody fragment can comprise a heavy chain constant region (Fe) of any suitable class, such as IgA, IgE, IgG, IgM, IgD, and sIgA. In some embodiments, the antibody or antibody fragment comprises a heavy chain constant region that is based upon wild-type IgG1, IgG2, or IgG4 antibodies, or variants thereof. It will be appreciated that each antibody class, or isotype, engages a distinct set of effector mechanisms for disposing of or neutralizing antigen once recognized. As such, in some embodiments, when the SARS-CoV-2 binding agent is an antibody or antibody fragment, it can exhibit one or more effector functions, such as participation in antibody-dependent complement-mediated lysis or antibody-dependent cellular toxicity via interactions with effector molecules and cells (e.g., activation of the complement system).

The SARS-CoV-2 binding agent also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

The SARS-CoV-2 binding agent also can be an intrabody or fragment thereof. An intrabody is an antibody which is expressed and which functions intracellularly. Intrabodies typically lack disulfide bonds (but can contain free cysteine side chains) and are capable of modulating the expression or activity of target genes through their specific binding activity. Intrabodies include single domain fragments such as isolated V_{H} and V_{L} domains, scFvs, VHH domains and fragments thereof. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The SARS-CoV-2 binding agent also can be an antibody conjugate. In this respect, the SARS-CoV-2 binding agent can be a conjugate of (1) an antibody, an alternative scaffold, or fragments thereof, and (2) a protein or non-protein moiety comprising the SARS-CoV-2 binding agent. the SARS-CoV-2 binding agent can be all or part of an antibody single variable domain conjugated to a peptide (e.g., PEG or serum albumin), a solid support (e.g., a substrate, a surface, a particle), a fluorescent molecule, a molecule or substance suitable for detection, e.g., a radiological agent or an imaging or dye agent or tag, other detection molecules or substances such as gold particles, a pharmaceutical agent, or a radiotherapy agent. Any method known in the art for separately conjugating an antigen-binding agent (e.g., an antibody) to a detectable moiety may be employed in the context of the invention (see, e.g., Hunter et al., Nature, 194: 495-496 (1962); David et al., Biochemistry, 13: 1014-1021 (1974); Pain et al., J. Immunol. Meth., 40: 219-230 (1981); and Nygren, J. Histochem. and Cytochem., 30: 407-412 (1982)

The SARS-CoV-2 binding agent can be, or can be obtained from, a human antibody, a non-human antibody, or a chimeric antibody. A “chimeric” antibody is an antibody or fragment thereof comprising both human and non-human regions. Preferably, the SARS-CoV-2 binding agent is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a camelid (e.g., camel, llama, alpaca, dromedary or guanaco) or rodent (e.g., a mouse or rat). A humanized antibody can comprise, one, two, or three CDRs (per heavy or light chain) obtained or derived from a non-human antibody. In one embodiment of the invention, CDRH1, CDRH2, and CDRH3 of the SARS-CoV-2 binding agent are obtained or derived from a camelid monoclonal antibody, e.g., a llama monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Kohler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, NY (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, New Jersey (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

### III. Nucleic Acids Encoding an SARS-CoV-2 Binding Agent and Cells Containing the Same

As used in this section “Nucleic Acids Encoding . . . ,” the term “antibody” or “antigen-binding fragment thereof” refers to any SARS-CoV-2 binding agent, e.g., any antibody or antigen-binding fragment, described herein. For the sake of clarity, this includes both antibodies or antigen-binding fragments thereof that comprise, consist of, or consist essentially of an immunoglobulin heavy chain polypeptide and an immunoglobulin light chain polypeptide, as well as single domain antibodies and antigen-binding fragments thereof.

One embodiment is a recombinant polynucleotide comprising, consisting of, or consisting essentially of a nucleotide sequence encoding the amino acid sequence of an SARS-CoV-2 binding agent disclosed herein (e.g., an antibody or antigen-binding fragment thereof). In one embodiment, the nucleotide sequence of the recombinant polynucleotide is codon optimized and/or codon pair optimized.

Another embodiment is a recombinant vector that comprises, consists of, or consists essentially of, a polynucleotide that encodes the amino acid sequence of an SARS-CoV-2 binding agent disclosed herein (e.g., an antibody or antigen-binding fragment thereof). The recombinant vector can be any suitable vector. Examples of suitable recombinant vectors include but are not limited to any suitable plasmid, e.g., pcDNA3.1, a pSV, a pCMV, a pBApo-CMV, a pBApo-EF1alpha expression vector, pET15a, and pSX2. In some embodiments, the recombinant vector is suitable for administration to a subject, preferably a mammalian subject, more preferably a human subject, such that the nucleic acid sequence that encodes the SARS-CoV-2 binding agent is expressed (e.g., in the form of mRNA) and/or the SARS-CoV-2 binding agent or any portion thereof is produced in the subject following administration. In some embodiments, the recombinant vector suitable for administration to a subject is a plasmid.

Yet another embodiment is an isolated cell that contains an SARS-CoV-2 binding agent or a recombinant polynucleotide that comprises, consists of, or consists essentially of a nucleic acid sequence that encodes the SARS-CoV-2 binding agent (e.g., an antibody or antigen-binding fragment thereof). In some embodiments, the isolated cell is suitable for administration to a subject, preferably a mammalian subject, more preferably a human subject, such that the nucleic acid sequence that encodes the SARS-CoV-2 binding agent is expressed (e.g., in the form of mRNA) and/or the SARS-CoV-2 binding agent or any portion thereof is produced in the subject following administration. Still another embodiment is a hybridoma or cell line that expresses a SARS-CoV-2 binding agent disclosed herein. One embodiment is a method for providing a SARS-CoV-2 binding agent, wherein the method comprises expressing in a cell in vitro or in vivo one or more nucleic acids encoding the SARS-CoV-2 binding agent. In some embodiments, the isolated cell is a eukaryotic cell. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Preferred yeast cells include, for example, Saccharomyces cerivisae and Pichia pastoris. In some embodiments, the hybridoma or cell line is mammalian. A number of suitable mammalian host cells are known in the art, and many are available from ATCC. Examples of suitable mammalian cells include, but are not limited to, Chinese hamster ovary cells (CHO) cells, such as CHO-K1 cells (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. Suitable cell lines also include hybridomas. Methods for selecting suitable mammalian host cells and methods for transformation, culture, amplification, screening, and purification of cells are known in the art.

The mammalian cell can be a human cell. For example, the mammalian cell can be a human lymphoid or lymphoid derived cell line, such as a cell line of pre-B lymphocyte origin. Examples of human lymphoid cells lines include, without limitation, RAMOS (CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof. In other embodiments, a prokaryotic organism is used, such as E. coli, or a bacillus strain.

### IV. SARS-CoV-2 Binding Agents and Compositions Including the Same

As used in this section “SARS-CoV-2 Binding Agents and . . . ,” the term “antibody” or “antigen-binding fragment thereof” refers to any SARS-CoV-2 binding agent, e.g., any antibody or antigen-binding fragment, described herein. For the sake of clarity, this includes both antibodies or antigen-binding fragments thereof that comprise, consist of, or consist essentially of an immunoglobulin heavy chain polypeptide and an immunoglobulin light chain polypeptide, as well as single domain antibodies and antigen-binding fragments thereof.

The SARS-CoV-2 binding agent can have any suitable affinity to SARS-CoV-2 or an epitope thereof. In some embodiments, the affinity is measured against a selected SARS-CoV-2 strain, e.g., Wuhan, Alpha, Beta, Delta, BA.2, BA.2.12.1, BA.3, BA.4, or BA.5, preferably BA.2, BA.4 or BA.5, more preferably BA.4 or BA.5, yet more preferably BA.5. The term “affinity” refers to the equilibrium constant for the reversible binding of two agents and is expressed as the dissociation constant (K_{D}). Affinity of a binding agent to a ligand, such as affinity of an antibody for an epitope, can be, for example, from about 1 femtomolar (fM) to about 100 micromolar (μM) (e.g., from about 1 femtomolar (fM) to about 1 nanomolar (nM), from about 1 nM to about 1 micromolar (μM), or from about 1 μM to about 100 μM). In one embodiment, the SARS-CoV-2 binding agent binds to SARS-CoV-2 BA.4, BA.5, BA.2, or BA.3, preferably BA.4 or BA.5 protein with a K_{D} less than or equal to 100 nM (e.g., 90 nM, 80 nM, 70 nM, 60 nM, 50 nM, 40 nM, 30 nM, 20 nM, 10 nM, 1 nM, 0.1 nM, 0.01 nM, 0.01 nM, or a range defined by any two of the foregoing values). In one embodiment, the SARS-CoV-2 binding agent can bind to SARS-CoV-2 protein with a K_{D} less than or equal to 1 nanomolar (e.g., 0.9 nM, 0.8 nM, 0.7 nM, 0.6 nM, 0.5 nM, 0.4 nM, 0.3 nM, 0.2 nM, 0.1 nM, 0.05 nM, 0.025 nM, 0.01 nM, 0.001 nM, or a range defined by any two of the foregoing values). In another embodiment, the SARS-CoV-2 binding agent can bind to SARS-CoV-2 with a K_{D} less than or equal to 200 pM (e.g., 190 pM, 175 pM, 150 pM, 125 pM, 110 pM, 100 pM, 90 pM, 80 pM, 75 pM, 60 pM, 50 pM, 40 pM, 30 pM, 25 pM, 20 pM, 15 pM, 10 pM, 5 pM, 1 pM, or a range defined by any two of the foregoing values). Immunoglobulin affinity for an antigen or epitope of interest can be measured using any art-recognized assay. Such methods include, for example, fluorescence activated cell sorting (FACS), separable beads (e.g., magnetic beads), surface plasmon resonance (SPR), solution phase competition (KINEXA™), antigen panning, competitive binding assays, and/or ELISA (see, e.g., Janeway et al. (eds.), Immunobiology, 5th ed., Garland Publishing, New York, NY, 2001).

The SARS-CoV-2 binding agent should have suitable stability in vivo. In one embodiment of the invention, the SARS-CoV-2 binding agent (e.g., an antibody or fragment thereof) has an in vivo half-life between about 30 minutes and 45 days (e.g., about 30 minutes, about 45 minutes, about 1 hour, about 2 hours, about 4 hours, about 6 hours, about 10 hours, about 12 hours, about 1 day, about 5 days, about 10 days, about 15 days, about 25 days, about 35 days, about 40 days, about 45 days, or a range defined by any two of the foregoing values). In another embodiment, the SARS-CoV-2 binding agent has an in vivo half-life between about 2 hours and 20 days (e.g., about 5 hours, about 10 hours, about 15 hours, about 20 hours, about 2 days, about 3 days, about 7 days, about 12 days, about 14 days, about 17 days, about 19 days, or a range defined by any two of the foregoing values). In another embodiment, the SARS-CoV-2 binding agent has an in vivo half-life between about 10 days and about 40 days (e.g., about 10 days, about 13 days, about 16 days, about 18 days, about 20 days, about 23 days, about 26 days, about 29 days, about 30 days, about 33 days, about 37 days, about 38 days, about 39 days, about 40 days, or a range defined by any two of the foregoing values).

The stability of the SARS-CoV-2 binding agent described herein also can be measured in terms of the transition mid-point value (Tₘ), which is the temperature where 50% of the amino acid sequence is in its native confirmation, and the other 50% is denatured. In general, the higher the Tₘ, the more stable the protein. In one embodiment of the invention, the SARS-CoV-2 binding agent comprises a transition mid-point value (Tₘ) in vitro of about 60-100 °C. For example, the SARS-CoV-2 binding agent can comprise a Tₘ in vitro of about 65-80 °C (e.g., 66 °C, 68 °C, 70 °C, 71 °C, 75 °C, or 79 °C), about 80-90 °C (e.g., about 81 °C, 85 °C, or 89 °C), or about 90-100 °C (e.g., about 91 °C, about 95 °C, or about 99 °C).

The stability of the SARS-CoV-2 binding agent can be measured using any other suitable assay known in the art, such as, for example, measuring serum half-life, differential scanning calorimetry (DSC), thermal shift assays, and pulse-chase assays. Other methods of measuring protein stability in vivo and in vitro that can be used in the context of the invention are described in, for example, Protein Stability and Folding, B. A. Shirley (ed.), Human Press, Totowa, New Jersey (1995); Protein Structure, Stability, and Interactions (Methods in Molecular Biology), Shiver J. W. (ed.), Humana Press, New York, NY (2010); and Ignatova, Microb. Cell Fact., 4: 23 (2005).

In some embodiments, the SARS-CoV-2 binding agent is formulated as a composition for administration to a subject (e.g., a mammal, such as a human or non-human primate) by any suitable route of administration, including parenteral, oral, intravenous, intraperitoneal, subcutaneous, pulmonary, transdermal, intramuscular, intranasal, buccal, sublingual, or suppository administration. The composition preferably is suitable for parenteral or intranasal administration. The term “parenteral,” as used herein, includes intravenous, intramuscular, subcutaneous, rectal, vaginal, and intraperitoneal administration. Parenteral administration may be, without limitation, by means of infusion (e.g., using an intravenous infusion system or pump) or injection (e.g., using a syringe). Intranasal administration may be, without limitation, by means of spray (e.g., using a spray bottle having a spray nozzle) or aerosolization (e.g., using a nebulizer or fluid aerosolization system). In some embodiments, the composition is administered to a mammal using peripheral systemic delivery by intravenous or subcutaneous injection. In some embodiments, the SARS-CoV-2 binding agent is administered by a single mode of administration, e.g., by intravenous infusion only. In some other embodiments, the SARS-CoV-2 binding agent is administered by more than one (multiple) modes of administration, e.g., by intravenous infusion and intranasal spray. Furthermore, the SARS-CoV-2 binding agent may be administered to an individual patient in one sitting (at a first time) only, or in multiple sittings (at a first time and at a subsequent second time). Examples of means of administration include, but are not limited to: a syringe, an intravenous infusion system, a spray bottle or spray system, a nebulizer, an aerosolization system, a swab, a capsule or pill, a transdermal delivery system, a controlled release delivery system, and any other protein delivery system. Formulations that include the SARS-CoV-2 binding agent may also include a stabilizing agent (a stabilizer), a preservation agent (a preservative), and other agents for stabilization and preservation. Other physical means, such as light-proof packaging and cold-storage, may also be used.

In some embodiments, the composition comprises the SARS-CoV-2 binding agent and at least one suitable carrier such as are well known in the art. The choice of carrier will be determined, in part, by the particular site to which the composition may be administered and the particular method used to administer the composition. The composition optionally can be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

The amount of SARS-CoV-2 binding agent (e.g., an antibody or antigen-binding fragment thereof) contained within the pharmaceutical compositions of the present invention may vary depending on the specific properties desired of the composition, as well as the particular circumstances and purposes for which the formulations are intended to be used. In certain embodiments the pharmaceutical compositions are liquid compositions that contain the antibody or antigen-binding fragment thereof in an amount from about 1 mg/mL to about 175 mg/mL (e.g., about 1 mg/mL, about 5 mg/mL, about 15 mg/mL, about 25 mg/mL, about 35 mg/mL, about 45 mg/mL, about 55 mg/mL, about 65 mg/mL, about 75 mg/mL, about 85 mg/mL, about 95 mg/mL, about 105 mg/mL, about 115 mg/mL, about 125 mg/mL, about 135 mg/mL, about 145 mg/mL, about 155 mg/mL, about 165 mg/mL, about 175 mg/mL, or a range defined by any two of the foregoing values), preferably from about 75 mg/mL to about 150 mg/mL (e.g., about 75 mg/mL, about 80 mg/mL, about 85 mg/mL, about 90 mg/mL, about 95 mg/mL, about 100 mg/mL, about 105 mg/mL, about 110 mg/mL, about 115 mg/mL, about 120 mg/mL, about 125 mg/mL, about 130 mg/mL, about 135 mg/mL, about 140 mg/mL, about 145 mg/mL, about 150 mg/mL, or a range defined by any two of the foregoing values), and more preferably from about 75 mg/mL to about 125 mg/mL.

In some embodiments, the SARS Co-V-2 binding agent e.g., an antibody or antigen-binding fragment thereof) is a part of kit. In some embodiments, the kit comprises, consists of, or consists essentially of a composition comprising, consisting of, or consisting essentially of a SARS Co-V-2 binding agent, a means of using the composition (e.g., a swab, an intranasal spray bottle, a dissolvable film for buccal or sublingual administration, or a microneedle array), and instructions for use.

### Methods Employing the SARS-CoV-2 Binding Agent, Including Methods of Prevention and Treatment

As used in this section “Methods Employing . . . ” the term “antibody” or “antigen-binding fragment thereof” refers to any SARS-CoV-2 binding agent, e.g., any antibody or antigen-binding fragment, described herein. For the sake of clarity, this includes both antibodies or antigen-binding fragments thereof that comprise, consist of, or consist essentially of an immunoglobulin heavy chain polypeptide and an immunoglobulin light chain polypeptide, as well as single domain antibodies and antigen-binding fragments thereof.

Provided herein is a method of preventing or treating a disease, disorder, or condition (e.g., COVID-19) in a mammal that is responsive to inhibition of SARS-CoV-2 binding to an ACE2 receptor, the method comprising inhibiting SARS-CoV-2 binding to an ACE2 receptor by administering to the mammal a SARS-CoV-2 binding agent disclosed herein, thereby preventing or treating the disease, disorder, or condition (e.g., COVID-19).

The subject can be a mammal, such as a human or non-human primate, with a disease, disorder, or condition in a mammal that is responsive to inhibition of SARS-CoV-2 binding to an ACE2 receptor, e.g., COVID-19.

As used herein, the terms “treatment,” “treating,” and the like refer to any indicia of success in the treatment or amelioration of an injury, pathology, condition (e.g., COVID-19), or symptom, including any objective or subjective parameter such as abatement; remission; diminishing of symptoms or making the symptom, injury, pathology, or condition more tolerable to the patient; reduction in the rate of symptom progression; decreasing the frequency or duration of the symptom or condition. The treatment or amelioration of symptoms can be based on any objective or subjective parameter, including, for example, the result of a physical examination. For example, the treatment reduces the severity or slows the onset or progression of one or more adverse symptoms of COVID-19 by any manner or degree. Reduction in adverse symptoms can be determined by any suitable technique.

As used herein, the terms “prevention,” “preventing,” “prophylactic,” and the like refer to the total or partial prevention of the onset of a disease (e.g., COVID-19) or one or more symptoms thereof. In some embodiments, “prevention” and the like refers to prevention of the infenction of a host with SARS-CoV-2. The prevention of infection and/or symptoms can be based on any objective or subjective parameter, including, for example, the result of a physical examination.

An embodiment of the invention is a method of preventing a disease, disorder, or condition (e.g., COVID-19) in a mammal that is responsive to inhibition of SARS-CoV-2 binding to an ACE2 receptor, the method comprising inhibiting SARS-CoV-2 binding to an ACE2 receptor by administering to the mammal a SARS-CoV-2 binding agent disclosed herein, thereby preventing, slowing, or delaying the onset of the disease, disorder, or condition (e.g., COVID-19), wherein the SARS-CoV-2 binding agent is administered intranasally.

The SARS-CoV-2 binding agent desirably exhibits one or more of the following biological activities: (a) inhibits the interaction between SARS-CoV-2 and an ACE2 receptor, and/or (b) inhibits endocytosis of SARS-CoV-2 into a cell. Other biological properties or characteristics of an antigen-binding agent recognized in the art include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or KINEXA™, in vitro or in vivo neutralization assays, receptor-ligand binding assays, cytokine or growth factor production and/or secretion assays, and signal transduction and immunohistochemistry assays.

The terms “inhibit” or “neutralize,” as used herein with respect to the activity of a SARS-CoV-2 binding agent, refer to the ability to substantially antagonize, prohibit, prevent, restrain, slow, disrupt, alter, eliminate, stop, or reverse the binding between SARS-CoV-2 and an ACE2 receptor and/or endocytosis of SARS-CoV-2 into a cell. For example, the SARS-CoV-2 binding agent preferably inhibits or neutralizes the binding and/or endocytosis of SARS-CoV-2 by at least about 20%, about 30%, about 40%, about 50%, about 60%, about 70%, about 80%, about 90%, about 95%, about 100%, or a range defined by any two of the foregoing values. In an embodiment, even partial (e.g., 50%) inhibition of binding of SARS-CoV-2 by the SARS-CoV-2 binding agent may provide a beneficial clinical effect in an individual by slowing or delaying the onset of disease (e.g., COVID-19 and/or associated symptoms), to thereby possibly enable the individual's body to develop and mount an innate immune response, for example, or to enable healthcare providers to provide the individual with additional medical support or options.

The SARS-CoV-2 binding agent can be used at dosages and for periods of time that are necessary to achieve a desired pharmacologic and/or physiologic effect (“therapeutically effective amount”). The therapeutically effective amount may vary according to factors such as the disease state, age, sex, weight of the individual, formulation of the SARS-CoV-2 binding agent and route of administration, as well as the ability of the SARS-CoV-2 binding agent to elicit a desired response in the individual. For example, a therapeutically effective amount of a SARS-CoV-2 binding agent can be an amount that inhibits binding of SARS-CoV-2 to an ACE2 receptor. In some embodiments, the dose is in the range of 1 pg/kg to 20 mg/kg of animal or human body weight; however, doses below or above this exemplary range are within the scope of the invention. The daily parenteral dose can be about 0.00001 μg/kg to about 20 mg/kg of total body weight (e.g., about 0.001 μg/kg, about 0.1 μg/kg, about 1 μg/kg, about 5 μg/kg, about 10 μg/kg, about 100 μg/kg, about 500 μg/kg, about 1 mg/kg, about 5 mg/kg, about 10 mg/kg, or a range defined by any two of the foregoing values), preferably from about 0.1 μg/kg to about 10 mg/kg of total body weight (e.g., about 0.5 μg/kg, about 1 μg/kg, about 50 μg/kg, about 150 μg/kg, about 300 μg/kg, about 750 μg/kg, about 1.5 mg/kg, about 5 mg/kg, or a range defined by any two of the foregoing values), more preferably from about 1 μg/kg to 5 mg/kg of total body weight (e.g., about 3 μg/kg, about 15 μg/kg, about 75 μg/kg, about 300 μg/kg, about 900 μg/kg, about 2 mg/kg, about 4 mg/kg, or a range defined by any two of the foregoing values), and even more preferably from about 0.5 to 15 mg/kg body weight per day (e.g., about 1 mg/kg, about 2.5 mg/kg, about 3 mg/kg, about 6 mg/kg, about 9 mg/kg, about 11 mg/kg, about 13 mg/kg, or a range defined by any two of the foregoing values).

In some embodiments the dose is about 20 mg or more (e.g., about 30 mg or more, about 50 mg or more, about 75 mg or more, or about 100 mg or more), and about 1000 mg or less (e.g., about 900 mg or less, about 800 mg or less, about 700 mg or less, about 600 mg or less, about 500 mg or less, about 400 mg or less, or about 300 mg or less) every 1-6 weeks (e.g., every week, every two weeks, every three weeks or every four weeks). In some embodiments, the SARS-CoV-2 binding agent is administered in a single loading does of about 1.5×-10× (e.g., about 2×-8×) the amount of following maintenance doses. Thus, for instance, the SARS-CoV-2 binding agent can be administered in a loading dose of about 200 mg-750 mg (e.g., about 300 mg-500 mg, or about 350 mg-450 mg) followed by a maintenance dose of about 50-300 mg (e.g., about 100 mg-300 mg or about 150 mg-250 mg) every two weeks or every month or two months (e.g., every 2-8 weeks or 2-4 weeks) thereafter as needed to effect or maintain a therapeutic response.

Therapeutic efficacy can be monitored by periodic assessment of treated patients. For repeated administrations over several days or longer, depending on the condition, the treatment can be repeated until a desired suppression of a disease, disorder or condition associated with SARS-CoV-2 infection occurs. However, other dosage regimens may be useful and are within the scope of the invention. The desired dosage can be delivered by a single bolus administration of the SARS-CoV-2 binding agent or composition described herein, by multiple bolus administrations of the SARS-CoV-2 binding agent composition described herein, or by continuous infusion administration of the SARS-CoV-2 binding agent or composition described herein, as examples. Other methods of administration are possible, such as intranasal (e.g., nasal spray) administration, oral administration, and other methods of administration. In one embodiment, oral administration is via a pill, capsule or lozenge, preferably a lozenge. Additionally, the SARS-CoV-2 binding agent may be administered in a first instance at a first time (e.g., as a single bolus administration, multiple bolus administrations, continuous intravenous, or intranasal administration), possibly followed by an intervening period of time (e.g., a pause or intermission in administration of the SARS-CoV-2 binding agent), and further followed by the administration of a (same or different formulation) SARS-CoV-2 binding agent in a second instance at a second time following the first time, and following any intervening period. For example, an individual may receive a first nasal spray administration of a first SARS-CoV-2 binding agent (as a nasal spray formulation), and then subsequently (e.g., after contracting COVID-19) the individual may receive a second intravenous administration of a second SARS-CoV-2 binding agent.

The SARS-CoV-2 binding agent of the invention may be administered alone or in combination with other drugs. For example, the SARS-CoV-2-binding agent can be administered in combination with other agents for the treatment or prevention of the disease, disorders, and conditions disclosed herein, or other agents known in the art to be useful for treating COVID-19.

Another method of the invention is a method for removing SARS-CoV-2 from a SARS-CoV-2-containing liquid, wherein the method comprises contacting the liquid with a SARS Co-2 binding agent disclosed herein, and removing the SARS-CoV-2 from the liquid. The contacting and removal steps can be undertaken using any suitable means known in the art. For example, immobilization of antibody or fragments onto solid support filters may be used to reduce or eliminate SARS-COV-2 from serum, e.g., during dialysis. Additionally, antibody or fragments may be administered intestinally to reduce or remove SARS-COV-2 from the intestinal lumen either as free antibody or immobilized antibody. In some embodiments, antibody or fragments thereof are affixed onto solid support beads or resins and used to purify SARS-COV-2 or SARS-CoV-2 spike containing virions, SARS-CoV-2 spike protein, or SARS-CoV-2 spike protein fragments (e.g., RBD peptides) from culture or expressing cells for use in diagnostics or vaccine production.

In another embodiment, the invention is a method of detecting SARS-CoV-2 in a sample, wherein the method comprises contacting the sample with a SARS-CoV-2 binding agent disclosed herein, and detecting the presence or absence of SARS-CoV-2 in the sample. Detection can be undertaken using any suitable means known in the art. For example, the SARS-CoV-2 binding agent can be affixed to a substrate and/or employed as part of an ELISA assay.

The invention further encompasses a SARS-CoV-2 binding agent or composition comprising the same, as described herein, for use to prevent or treat a disease, disorder, or condition in a mammal that is responsive to inhibition of SARS-CoV-2 binding to an ACE2 receptor (e.g., COVID-19) in accordance with the methods provided herein. In some embodiments, the SARS-CoV-2 binding agent comprises an immunoglobulin heavy chain variable region of any one of SEQ ID NO: 1-4, or at least the CDRs thereof, as described herein.

Certain sequences referenced herein are set forth in the following Tables 1 and 2. Table 1 is a chart depicting the amino acid sequence of SEQ ID NO: 1 and also possible alternative residues for certain residues (as captured in SEQ ID NO: 2), wherein the first listed (left-most) alternative residue is the most preferred alternative residue. Table 2 lists certain sequences referenced herein.

TABLE 1

| Residue | | Possible alternative residues for | | | | | | | |
|---|---|---|---|---|---|---|---|---|---|
| Number of | SEQ | certain residues as shown in | | | | | | | |
| Sequence | ID | SEQ ID NO: 2, wherein the first | | | | | | | |
| of SEQ | NO: | listed (left-most) residue is most | | | | | | | |
| ID NO: 1 | 1 | preferred alternative | | | | | | | |
| 1 | Q | E | | | | | | | |
| 2 | V | | | | | | | | |
| 3 | Q | K | E | | | | | | |
| 4 | L | | | | | | | | |
| 5 | Q | V | A | | | | | | |
| 6 | E | A | Q | | | | | | |
| 7 | S | T | | | | | | | |
| 8 | G | | | | | | | | |
| 9 | G | | | | | | | | |
| 10 | G | D | | | | | | | |
| 11 | L | S | C | | | | | | |
| 12 | V | | | | | | | | |
| 13 | Q | R | E | | | | | | |
| 14 | T | A | P | | | | | | |
| 15 | G | | | | | | | | |
| 16 | G | D | E | | | | | | |
| 17 | S | | | | | | | | |
| 18 | L | | | | | | | | |
| 19 | R | T | S | | | | | | |
| 20 | L | | | | | | | | |
| 21 | S | | | | | | | | |
| 22 | C | | | | | | | | |
| 23 | A | V | T | K | | | | | |
| 24 | A | V | T | | | | | | |
| 25 | S | | | | | | | | |
| 26 | G | A | V | R | | | | | |
| 27 | S | R | T | L | D | | | | |
| 28 | F | T | I | | | | | | |
| 29 | F | V | L | S | | | | | |
| 30 | S | R | Q | N | | | | | |
| 31 | I | S | T | F | L | K | N | E | V |
| 32 | N | Y | I | F | G | | | | |
| 33 | D | T | S | A | Q | R | V | | |
| 34 | M | V | I | | | | | | |
| 35 | G | R | T | Y | A | N | | | |
| 36 | W | | | | | | | | |
| 37 | Y | F | | | | | | | |
| 38 | R | | | | | | | | |
| 39 | Q | | | | | | | | |
| 40 | A | T | | | | | | | |
| 41 | P | | | | | | | | |
| 42 | G | | | | | | | | |
| 43 | K | T | S | | | | | | |
| 44 | Q | E | | | | | | | |
| 45 | R | | | | | | | | |
| 46 | E | | | | | | | | |
| 47 | L | F | | | | | | | |
| 48 | V | | | | | | | | |
| 49 | A | S | | | | | | | |
| 50 | M | A | T | S | | | | | |
| 51 | I | S | T | R | | | | | |
| 52 | T | V | Q | N | S | | | | |
| 53 | G | S | D | T | N | A | W | | |
| 54 | D | G | S | I | | | | | |
| 55 | D | G | Y | R | | | | | |
| 56 | S | R | N | A | | | | | |
| 57 | T | I | | | | | | | |
| 58 | N | Y | D | T | S | K | R | H | |
| 59 | Y | H | | | | | | | |
| 60 | A | E | D | V | I | T | S | K | |
| 61 | D | E | | | | | | | |
| 62 | S | F | | | | | | | |
| 63 | V | A | | | | | | | |
| 64 | K | T | E | | | | | | |
| 65 | G | D | | | | | | | |
| 66 | R | | | | | | | | |
| 67 | F | V | | | | | | | |
| 68 | T | A | | | | | | | |
| 69 | I | A | | | | | | | |
| 70 | S | | | | | | | | |
| 71 | R | G | | | | | | | |
| 72 | D | | | | | | | | |
| 73 | N | T | F | D | | | | | |
| 74 | L | A | V | M | S | D | | | |
| 75 | K | Q | E | | | | | | |
| 76 | N | T | G | H | A | | | | |
| 77 | T | L | I | | | | | | |
| 78 | V | D | | | | | | | |
| 79 | Y | S | H | D | | | | | |
| 80 | L | | | | | | | | |
| 81 | Q | D | E | | | | | | |
| 82 | M | L | V | | | | | | |
| 83 | N | S | T | | | | | | |
| 84 | S | D | N | | | | | | |
| 85 | L | | | | | | | | |
| 86 | K | E | | | | | | | |
| 87 | P | V | S | | | | | | |
| 88 | E | K | D | | | | | | |
| 89 | D | | | | | | | | |
| 90 | T | | | | | | | | |
| 91 | A | | | | | | | | |
| 92 | L | V | | | | | | | |
| 93 | Y | | | | | | | | |
| 94 | Y | R | T | | | | | | |
| 95 | C | | | | | | | | |
| 96 | N | A | H | K | V | T | | | |
| 97 | A | R | Y | P | K | V | T | | |
| 98 | E | D | A | T | K | R | S | | |
| 99 | V | R | G | K | P | L | D | F | |
| 100 | K | G | Y | S | R | D | E | | |
| 101 | V | Y | F | W | G | I | P | | |
| 102 | I | V | Y | F | S | Q | R | | |
| 103 | I | V | L | S | T | Y | C | | |
| 104 | W | Y | F | L | V | T | P | | |
| 105 | D | R | A | P | S | C | | | |
| 106 | S | A | T | P | C | Q | D | W | |
| 107 | R | K | D | E | T | P | Y | | |
| 108 | F | D | W | K | Y | S | | | |
| 109 | N | S | D | E | F | Y | | | |
| 110 | Y | D | E | N | A | G | | | |
| 111 | Y | E | V | S | T | | | | |
| 112 | W | | | | | | | | |
| 113 | G | | | | | | | | |
| 114 | Q | K | | | | | | | |
| 115 | G | | | | | | | | |
| 116 | T | | | | | | | | |
| 117 | Q | E | R | L | P | | | | |
| 118 | V | | | | | | | | |
| 119 | T | | | | | | | | |
| 120 | V | | | | | | | | |
| 121 | S | | | | | | | | |
| 122 | S | | | | | | | | |

TABLE 2

| SEQ | | |
|---|---|---|
| ID | | |
| NO | Description | Sequence |
| 1 | Heavy | QVQLQESGGGLVQTGGSLRLSCAASGSFFSINDMGWYRQAPGKQRELVAMITGDDSTNYADSVKGRF |
| | Chain | TISRDNLKNTVYLQMNSLKPEDTALYYCNAEVKVIIWDSRFNYYWGQGTQVTVSS |
| | Variable | |
| | Region | |
| | Sequence | |
| 2 | Heavy | X1 V X2 L X3 X4 X5 G G X6 X7 V X8 X9 G X10 SL |
| | Chain | X11 L SC X12 X13 S X14 X15 X16 X17 X18 X19 |
| | Variable | X20 X21 X22 X23 W X24 RQX25 PGX26 X27 RE X28 |
| | Region | V X29 X30 X31 X32 X33 X34 X35 X36 X37 X38 |
| | Sequence | X39 X40 X41 X42 X43 X44 X45 R X46 X47 X48 S |
| | | X49 D X50 X51 X52 X53 X54 X55 X56 L X57 X58 X59 |
| | | X60 L X61 X62 X63 D TA X64 Y X65 C X66 X67 |
| | | X68 X69 X70 X71 X72 X73 X74 X75 X76 X77 X78 X79 |
| | | X80 X81 W GX82 GTX83 VTV S S |
| | | (*see below for information regarding the |
| | | identity of the X residues) |
| 3 | Heavy | QVQLQESGGGLVQTGGSLRLSCAASGSFFSINDMGWYRQAPGKQRELVAMITGDDSTNYADSVKGRF |
| | Chain | TISRDNLKNTVYLQMNSLKPEDTALYYCNAEVKVIIWDSRFNYYWGQGTQVTVSSSGGGGDGGGGSG |
| | Variable | DKTHTCPPCPAPELLGGPSVFLFPPKPKDTLMISRTPEVTCVVVDVSHEDPEVKFNWYVDGVEVHNAK |
| | Region | TKPREEQYNSTYRVVSVLTVLHQDWLNGKEYKCKVSNKALPAPIEKTISKAKGQPREPQVYTLPPSREE |
| | Sequence | LTKNQVSLTCLVKGFYPSDIAVEWESNGQPENNYKTTPPVLDSDGSFFLYSKLTVDKSRWQQGNVFSC |
| | and Human | SVMHEALHNHYTQKSLSLSPGS |
| | IgG1 Fc | |
| | Sequence | |
| 4 | Heavy | X1 V X2 L X3 X4 X5 GG X6 X7 V X8 X9 GX10 SL X11 L |
| | Chain | SC X12 X13 S X14 X15 X16 X17 X18 X19 X20 X21 X22 |
| | Variable | X23 W X24 RQX25PGX26 X27 RE X28 V X29 X30 X31 X32 |
| | Region | X33 X34 X35 X36 X37 X38 X39 X40 X41 X42 X43 X44 X45 |
| | Sequence | R X46 X47 X48 S X49 D X50 X51 X52 X53 X54 X55 X56 |
| | and Human | L X57 X58 X59 X60 L X61 X62 X63 D TA X64 Y X65 C |
| | IgG1 Fc | X66 X67 X68 X69 X70 X71 X72 X73 X74 X75 X76 X77 |
| | Sequence | X78 X79 X80 X81 WG X82 GTX83 V TV SS |
| | | SGGGGDGGGGSGDKTHTCPPCPAPELLGGPSVFLFPPKPKDTLMISRTPEVTCVVVDVSH |
| | | EDPEVKFNWYVDGVEVHNAKTKPREEQYNSTYRVVSVLTVLHQDWLNGKEYKCKVSNKAL |
| | | PAPIEKTISKAKGQPREPQVYTLPPSREELTKNQVSLTCLVKGFYPSDIAVEWESNGQPE |
| | | NNYKTTPPVLDSDGSFFLYSKLTVDKSRWQQGNVFSCSVMHEALHNHYTQKSLSLSPGS |
| | | (*see below for information regarding the identity of |
| | | the X residues) |
| 5 | CDR1 | GSFFSIN |
| | (Chothia) | |
| 6 | CDR1 | INDMG |
| | (Kabat) | |
| 7 | CDR1 | GSFFSIND |
| | (IGMT) | |
| 8 | CDR1 | X1 X2 X3 X4 X5 X6 X7; |
| | (Chothia) | wherein X1 is glycine (G), alanine (A), valine (V), or arginine (R); |
| | | X2 is serine (S), arginine (R), threonine (T), leucine (L), |
| | | or aspartic acid (D); |
| | | X3 is phenylalanine (F), threonine (T), or isoleucine (I); |
| | | X4 is phenylalanine (F), valine (V), leucine (L), or serine (S); |
| | | X5 is serine (S), arginine (R), glutamine (Q), or asparagine (N); |
| | | X6 is isoleucine (I), serine (S), threonine (T), phenylalanine (F), |
| | | leucine (L), lysine (K), asparagine (N), glutamic acid (E), or valine (V),; |
| | | and X7 is asparagine (N), tyrosine (Y), , isoleucine (I), |
| | | phenylalanine (F), or glycine (G). |
| 9 | CDR1 | X1 X2 X3 X4 X5; |
| | (Kabat) | wherein X1 is isoleucine (I), serine (S), threonine (T), phenylalanine (F), |
| | | leucine (L), lysine (K), asparagine (N), glutamic acid (E), or valine (V); |
| | | X2 is asparagine (N), tyrosine (Y), isoleucine (I), phenylalanine (F), or |
| | | glycine (G); |
| | | X3 is aspartic acid (D), threonine (T), serine (S), alanine (A), glutamine |
| | | (Q), arginine (R), or valine (V); |
| | | X4 is methionine (M), valine (V), or isoleucine (I), and X5 is glycine (G), |
| | | arginine (R), threonine (T), tyrosine (Y), alanine (A), or asparagine (N). |
| 10 | CDR1 | X1 X2 X3 X4 X5 X6 X7 X8; |
| | (IGMT) | wherein X1 is glycine (G), alanine (A), valine (V), or arginine (R); |
| | | X2 is serine (S), arginine (R), threonine (T), leucine (L), |
| | | or aspartic acid (D); |
| | | X3 is phenylalanine (F), threonine (T), or isoleucine (I); |
| | | X4 is phenylalanine (F), valine (V), leucine (L), or serine (S); |
| | | X5 is serine (S), arginine (R), glutamine (Q), or asparagine (N); |
| | | X6 is isoleucine (I), serine (S), threonine (T), phenylalanine (F), |
| | | leucine (L), lysine (K), asparagine (N), glutamic acid (E), or valine (V); |
| | | X7 is asparagine (N), tyrosine (Y), isoleucine (I), phenylalanine (F), |
| | | or glycine (G); |
| | | and X8 is is aspartic acid (D), threonine (T), serine (S), alanine (A), |
| | | glutamine (Q), arginine (R), or valine (V). |
| 11 | CDR2 | TGDDS |
| | (Chothia) | |
| 12 | CDR2 | MITGDDSTNYADSVKG |
| | (Kabat) | |
| 13 | CDR2 | ITGDDST |
| | (IGMT) | |
| 14 | CDR2 | X1 X2 X3 X4 X5; wherein X1 is threonine (T), valine (V), |
| | (Chothia) | glutamine (Q), asparagine (N), or serine (S); |
| | | X2 is glycine (G), serine (S), aspartic acid (D), threonine (T), |
| | | asparagine (N), alanine (A), or tryptophan (W); |
| | | X3 is aspartic acid (D), glycine (G), serine (S), or isoleucine (I); |
| | | X4 is aspartic acid (D), glycine (G), tyrosine (Y), or |
| | | arginine (R); and |
| | | X5 is serine (S), arginine (R), asparagine (N), or alanine (A). |
| 15 | CDR2 | X1 X2 X3 X4 X5 X6 X7 X8 X9 X10 X11 X12 X13 X14 X15 X16, |
| | (Kabat) | wherein X1 is methionine (M), alanine (A), |
| | | threonine (T), or serine (S); X2 is isoleucine (I), serine (S), |
| | | threonine (T), or arginine (R); X3 is threonine (T), |
| | | valine (V), glutamine (Q), asparagine (N), or serine (S); |
| | | X4 is glycine (G), serine (S), aspartic acid (D), |
| | | threonine (T), asparagine (N), alanine (A), or tryptophan (W); |
| | | X5 is aspartic acid (D), glycine (G), serine (S), or |
| | | isoleucine (I); X6 is aspartic acid (D), glycine (G), |
| | | tyrosine (Y), or arginine (R); X7 is serine (S), arginine (R), |
| | | asparagine (N), or alanine (A); X8 is threonine (T) or |
| | | isoleucine (I); X9 is asparagine (N), tyrosine (Y), aspartic |
| | | acid (D), threonine (T), serine (S), lysine (K), arginine (R), |
| | | or histidine (H); X10 is tyrosine (Y)or histidine (H); |
| | | X11 is alanine (A), glutamic acid (E), aspartic acid (D), |
| | | valine (V), isoleucine (I), threonine (T), serine (S), or |
| | | lysine (K); X12 is aspartic acid (D) or glutamic acid (E); |
| | | X13 is serine (S) or phenylalanine (F); X14 is valine |
| | | (V) or alanine (A); X15 is lysine (K), threonine (T), or |
| | | glutamic acid (E); and X16 is glycine (G) or aspartic acid |
| | | (D). |
| 16 | CDR2 | X1 X2 X3 X4 X5 X6 X7; |
| | (IGMT) | wherein X1 is isoleucine (I), serine (S), threonine (T), |
| | | or arginine (R); X2 is threonine |
| | | (T), valine (V), glutamine (Q), asparagine (N), or serine |
| | | (S); X3 is glycine (G), serine (S), aspartic acid (D), |
| | | threonine (T), asparagine (N), alanine (A), or tryptophan |
| | | (W); X4 is aspartic acid (D), glycine (G), serine (S), or |
| | | isoleucine (I); X5 is aspartic acid (D), glycine (G), |
| | | tyrosine (Y), or arginine (R); X6 is serine (S), arginine (R), |
| | | asparagine (N), or alanine (A); and X7 is threonine (T) |
| | | or isoleucine (I). |
| 17 | CDR3 | EVKVIIWDSRFNYY |
| | (Chothia & | |
| | Kabat) | |
| 18 | CDR3 | NAEVKVIIWDSRFNYY |
| | (IGMT) | |
| 19 | CDR3 | X1 X2 X3 X4 X5 X6 X7 X8 X9 X10 X11 X12 X13 X14; |
| | (Chothia & | wherein X1 is glutamic acid (E), aspartic acid (D), |
| | Kabat) | alanine (A), threonine (T), lysine (K), arginine (R), |
| | | or serine (S); X2 is valine (V), arginine (R), glycine (G), |
| | | lysine (K), proline (P), leucine (L), aspartic acid (D), |
| | | or phenylalanine (F); X3 is lysine (K), glycine (G), |
| | | tyrosine (Y), serine (S), arginine (R), aspartic acid (D), |
| | | or glutamic acid (E); X4 is valine (V), tyrosine (Y), |
| | | phenylalanine (F), tryptophan (W), glycine (G), isoleucine |
| | | (I), or proline (P); X5 is isoleucine (I), valine (V), |
| | | tyrosine (Y), phenylalanine (F), serine (S), glutamine (Q), |
| | | or arginine (R); X6 is isoleucine (I), valine (V), |
| | | leucine (L), serine (S), threonine (T), tyrosine (Y), or |
| | | cysteine (C); X7 is tryptophan (W), tyrosine (Y), |
| | | phenylalanine (F), leucine (L), valine (V), threonine (T), |
| | | or proline (P); X8 is aspartic acid (D), arginine (R), |
| | | alanine (A), proline (P), serine (S), or cysteine (C); |
| | | X9 is serine (S), alanine (A), threonine (T), proline (P), |
| | | cysteine (C), glutamine (Q), aspartic acid (D), or |
| | | tryptophan (W); X10 is arginine (R), lysine (K), aspartic acid |
| | | (D), glutamic acid (E), threonine (T), proline (P), or |
| | | tyrosine (Y); X11 is phenylalanine (F), aspartic acid (D), |
| | | tryptophan (W), lysine (K), tyrosine (Y), or serine (S); |
| | | X12 is asparagine (N), serine (S), aspartic acid (D), |
| | | glutamic acid (E), phenylalanine (F), or tyrosine (Y); |
| | | X13 is tyrosine (Y), aspartic acid (D), glutamic acid (E), |
| | | asparagine (N), alanine (A), or glycine (G); and X14 is |
| | | tyrosine (Y), glutamic acid (E), valine (V), serine (S), or |
| | | threonine (T). |
| 20 | CDR3 | X1 X2 X3 X4 X5 X6 X7 X8 X9 X10 X11 X12 X13 X14 X15 X16; |
| | (IGMT) | wherein X1 is asparagine (N), alanine (A), |
| | | histidine (H), lysine (K), valine (V), or threonine (T); |
| | | X2 is alanine (A), arginine (R), tyrosine (Y), proline (P), |
| | | lysine (K), valine (V), or threonine (T); |
| | | X3 is glutamic acid (E), aspartic acid (D), alanine (A), |
| | | threonine (T), |
| | | lysine (K), arginine (R), or serine (S); |
| | | X4 is valine (V), arginine (R), glycine (G), lysine (K), |
| | | proline (P), leucine (L), aspartic acid (D), or |
| | | phenylalanine (F); |
| | | X5 is lysine (K), glycine (G), tyrosine (Y), serine (S), arginine (R), |
| | | aspartic acid (D), or glutamic acid (E); |
| | | X6 is valine (V), tyrosine (Y), phenylalanine (F), tryptophan (W), |
| | | glycine (G), isoleucine (I), or proline (P); |
| | | X7 is isoleucine (I), valine (V), tyrosine (Y), |
| | | phenylalanine (F), serine |
| | | (S), glutamine (Q), or arginine (R); |
| | | X8 is isoleucine (I), valine (V), leucine (L), |
| | | serine (S), threonine (T), |
| | | tyrosine (Y), or cysteine (C); |
| | | X9 is tryptophan (W), tyrosine (Y), phenylalanine (F), |
| | | leucine (L), valine (V), |
| | | threonine (T), or proline (P); |
| | | X10 is aspartic acid (D), arginine (R), alanine (A), |
| | | proline (P), serine (S), or |
| | | cysteine (C); |
| | | X11 is serine (S), alanine (A), threonine (T), proline (P), |
| | | cysteine (C), glutamine (Q), aspartic acid |
| | | (D), or tryptophan (W); X12 is arginine (R), lysine (K), |
| | | aspartic acid (D), glutamic acid (E), threonine (T), |
| | | proline (P), or tyrosine (Y); |
| | | X13 is phenylalanine (F), aspartic acid (D), |
| | | tryptophan (W), lysine (K), tyrosine |
| | | (Y), or serine (S); |
| | | X14 is asparagine (N), serine (S), aspartic acid (D), |
| | | glutamic acid (E), phenylalanine (F), or |
| | | tyrosine (Y); |
| | | X15 is tyrosine (Y), aspartic acid (D), glutamic |
| | | acid (E), asparagine (N), alanine (A), or glycine |
| | | (G); and |
| | | X16 is tyrosine (Y), glutamic acid (E), valine (V), |
| | | serine (S), or threonine (T). |

*With regard to SEQ ID NOs: 2 and 4, the “X[number]” residues are defined as follows: wherein (a) X1 is glutamine (Q) or glutamic acid (E), (b) X2 is glutamine (Q), lysine (K), or glutamic acid (E), (c) X3 is glutamine (Q), valine (V), or alanine (A), (d) X4 is glutamic acid (E), alanine (A), or glutamine (Q), (e) X5 is serine (S) or threonine (T), (f) X6 is glycine (G) or aspartic acid (D), (g) X7 is leucine (L), serine (S), or cysteine (C), (h) X8 is glutamine (Q), arginine (R), or glutamic acid (E), (i) X9 is threonine (T), alanine (A), or proline (P), (j) X10 is glycine (G), aspartic acid (D), or glutamic acid (E), (k) X11 is arginine (R), threonine (T), or serine (S), (1) X12 is alanine (A), valine (V), threonine (T), or lysine (K), (m) X13 is alanine (A), valine (V), or threonine (T), (n) X14 is glycine (G), alanine (A), valine (V), or arginine (R), (o) X15 is serine (S), arginine (R), threonine (T), leucine (L), or aspartic acid (D), (p) X16 is phenylalanine (F), threonine (T), or isoleucine (I), (q) X17 is phenylalanine (F), valine (V), leucine (L), or serine (S), (r) X18 is serine (S), arginine (R), glutamine (Q), or asparagine (N), (s) X19 is isoleucine (I), serine (S), threonine (T), phenylalanine (F), leucine (L), lysine (K), asparagine (N), glutamic acid (E), or valine (V), (t) X20 is asparagine (N), tyrosine (Y), isoleucine (I), phenylalanine (F), or glycine (G), (u) X21 is aspartic acid (D), threonine (T), serine (S), alanine (A), glutamine (Q), arginine (R), or valine (V), (v) X22 is methionine (M), valine (V), or isoleucine (I), (w) X23 is glycine (G), arginine (R), threonine (T), tyrosine (Y), alanine (A), or asparagine (N), (x) X24 is tyrosine (Y) or phenylalanine (F), (y) X25 is alanine (A) or threonine (T), (z) X26 is lysine (K), threonine (T), or serine (S), (aa) X27 is glutamine (Q) or glutamic acid (E), (ab) X28 is leucine (L) or phenylalanine (F), (ac) X29 is alanine (A) or serine (S), (ad) X30 is methionine (M), alanine (A), threonine (T), or serine (S), (ae) X31 is isoleucine (I), serine (S), threonine (T), or arginine (R), (af) X32 is threonine (T), valine (V), glutamine (Q), asparagine (N), or serine (S), (ag) X33 is glycine (G), serine (S), aspartic acid (D), threonine (T), asparagine (N), alanine (A), or tryptophan (W), (ah) X34 is aspartic acid (D), glycine (G), serine (S), or isoleucine (I), (ai) X35 is aspartic acid (D), glycine (G), tyrosine (Y), or arginine (R), (aj) X36 is serine (S), arginine (R), asparagine (N), or alanine (A), (ak) X37 is threonine (T) or isoleucine (I), (al) X38 is asparagine (N), tyrosine (Y), aspartic acid (D), threonine (T), serine (S), lysine (K), arginine (R), or histidine (H), (am) X39 is tyrosine (Y)or histidine (H), (an) X40 is alanine (A), glutamic acid (E), aspartic acid (D), valine (V), isoleucine (I), threonine (T), serine (S), or lysine (K), (ao) X41 is aspartic acid (D) or glutamic acid (E), (ap) X42 is serine (S) or phenylalanine (F), (aq) X43 is valine (V) or alanine (A), (ar) X44 is lysine (K), threonine (T), or glutamic acid (E), (as) X45 is glycine (G) or aspartic acid (D), (at) X46 is phenylalanine (F) or valine (V), (au) X47 is threonine (T) or alanine (A), (av) X48 is isoleucine (I) or alanine (A), (aw) X49 is arginine (R) or glycine (G), (ax) X50 is asparagine (N), threonine (T), phenylalanine (F), or aspartic acid (D), (ay) X51 is leucine (L), alanine (A), valine (V), methionine (M), serine (S), or aspartic acid (D), (az) X52 is lysine (K), glutamine (Q), or glutamic acid (E), (ba) X53 is asparagine (N), threonine (T), glycine (G), histidine (H), or alanine (A), (bb) X54 is threonine (T), leucine (L), or isoleucine (I), (bc) X55 is valine (V) or aspartic acid (D), (bd) X56 is tyrosine (Y), serine (S), histidine (H), or aspartic acid (D), (be) X57 is glutamine (Q), aspartic acid (D), or glutamic acid (E), (bf) X58 is methionine (M), leucine (L), or valine (V), (bg) X59 is asparagine (N), serine (S), or threonine (T), (bh) X60 is serine (S), aspartic acid (D), or asparagine (N), (bi) X61 is lysine (K) or glutamic acid (E), (bj) X62 is proline (P), valine (V), or serine (S), (bk) X63 is glutamic acid (E), lysine (K), or aspartic acid (D), (bl) X64 is leucine (L) or valine (V), (bm) X65 is tyrosine (Y), arginine (R), or threonine (T), (bn) X66 is asparagine (N), alanine (A), histidine (H), lysine (K), valine (V), or threonine (T), (bo) X67 is alanine (A), arginine (R), tyrosine (Y), proline (P), lysine (K), valine (V), or threonine (T), (bp) X68 is glutamic acid (E), aspartic acid (D), alanine (A), threonine (T), lysine (K), arginine (R), or serine (S), (bq) X69 is valine (V), arginine (R), glycine (G), lysine (K), proline (P), leucine (L), aspartic acid (D), or phenylalanine (F), (br) X70 is lysine (K), glycine (G), tyrosine (Y), serine (S), arginine (R), aspartic acid (D), or glutamic acid (E), (bs) X71 is valine (V), tyrosine (Y), phenylalanine (F), tryptophan (W), glycine (G), isoleucine (I), or proline (P), (bt) X72 is isoleucine (I), valine (V), tyrosine (Y), phenylalanine (F), serine (S), glutamine (Q), or arginine (R), (bu) X73 is isoleucine (I), valine (V), leucine (L), serine (S), threonine (T), tyrosine (Y), or cysteine (C), (bv) X74 is tryptophan (W), tyrosine (Y), phenylalanine (F), leucine (L), valine (V), threonine (T), or proline (P), (bw) X75 is aspartic acid (D), arginine (R), alanine (A), proline (P), serine (S), or cysteine (C), (bx) X76 is serine (S), alanine (A), threonine (T), proline (P), cysteine (C), glutamine (Q), aspartic acid (D), or tryptophan (W), (by) X77 is arginine (R), lysine (K), aspartic acid (D), glutamic acid (E), threonine (T), proline (P), or tyrosine (Y), (bz) X78 is phenylalanine (F), aspartic acid (D), tryptophan (W), lysine (K), tyrosine (Y), or serine (S), (ca) X79 is asparagine (N), serine (S), aspartic acid (D), glutamic acid (E), phenylalanine (F), or tyrosine (Y), (cb) X80 is tyrosine (Y), aspartic acid (D), glutamic acid (E), asparagine (N), alanine (A), or glycine (G), (cc) X81 is tyrosine (Y), glutamic acid (E), valine (V), serine (S), or threonine (T), (cd) X82 is glutamine (Q) or lysine (K), (ce) X83 is glutamine (Q), glutamic acid (E), arginine (R), leucine (L), or proline (P).

Aspects, including embodiments, of the subject matter described herein may be beneficial alone or in combination, with one or more other aspects or embodiments. Without limiting the foregoing description, certain non-limiting aspects of the disclosure numbered 1-26 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. A SARS-CoV-2 binding agent comprising a single domain variable region polypeptide, wherein:
the single domain variable region polypeptide comprises a complementarity determining region 1 (CDR1) comprising SEQ ID NO: 5, a complementarity determining region 2 (CDR2) comprising SEQ ID NO: 12, and a complementarity determining region 3 (CDR3) comprising SEQ ID NO: 17, as determined via Chothia;
the single domain variable region polypeptide comprises a complementarity determining region 1 (CDR1) comprising SEQ ID NO: 6, a complementarity determining region 2 (CDR2) comprising SEQ ID NO: 11, and a complementarity determining region 3 (CDR3) comprising SEQ ID NO: 17, as determined via Kabat; or
the single domain variable region polypeptide comprises a complementarity determining region 1 (CDR1) comprising SEQ ID NO: 7, a complementarity determining region 2 (CDR2) comprising SEQ ID NO: 13, and a complementarity determining region 3 (CDR3) comprising SEQ ID NO: 18, as determined via IGMT.

2. A SARS-CoV-2 binding agent comprising an immunoglobulin heavy chain variable region polypeptide, wherein:
the immunoglobulin heavy chain variable region polypeptide comprises a complementarity determining region 1 (HCDR1) comprising SEQ ID NO: 5, a complementarity determining region 2 (HCDR2) comprising SEQ ID NO: 12, and a complementarity determining region 3 (HCDR3) comprising SEQ ID NO: 17, as determined via Chothia;
the immunoglobulin heavy chain variable region polypeptide comprises a complementarity determining region 1 (HCDR1) comprising SEQ ID NO: 6, a complementarity determining region 2 (HCDR2) comprising SEQ ID NO: 11, and a complementarity determining region 3 (HCDR3) comprising SEQ ID NO: 17, as determined via Kabat; or
the immunoglobulin heavy chain variable region polypeptide comprises a complementarity determining region 1 (HCDR1) comprising SEQ ID NO: 7, a complementarity determining region 2 (HCDR2) comprising SEQ ID NO: 13, and a complementarity determining region 3 (HCDR3) comprising SEQ ID NO: 18, as determined via IGMT.

3. A SARS-CoV-2 binding agent comprising an immunoglobulin heavy chain variable region or single domain variable region of any one of SEQ ID NOs: 1 and 2.

4. The SARS-CoV-2 binding agent of aspect 3, wherein the immunoglobulin heavy chain variable region or single domain variable region of any one of SEQ ID NOs: 1 or 2 comprises CDR sequences, and wherein the SARS-CoV-2 binding agent comprises at least the CDR sequences.

5. A SARS-CoV-2 binding agent comprising an immunoglobulin heavy chain variable region or single domain variable region comprising at least a complementarity determining region 3 (CDR3) comprising any one of SEQ ID NOs 17 and 18.

6. The SARS-CoV-2 binding agent of any one of aspects 1-5 comprising an immunoglobulin heavy chain variable region polypeptide or a single domain variable region polypeptide that comprises an amino acid sequence that is at least 80%, at least 85%, at least 90%, at least 91%, at least 92%, at least 93%, at least 94%, at least 95%, at least 96%, at least 97%, at least 98%, or at least 99% identical to any one of SEQ ID NOs: 1-4.

7. The SARS-CoV-2 binding agent of any one of aspects 1-5 comprising an immunoglobulin heavy chain variable region polypeptide or a single domain variable region polypeptide that comprises an amino acid sequence identical to any one of SEQ ID NOs: 1-4, except that the amino acid sequence differs at 1, 2, 3, 4, 5, 6, 7, 8, 9, 10, 11, 12, 13, 14, 15, 16, 17, 18, 19, or 20 residues, preferably at only 1, 2, 3, 4, 5, 6, 7, 8, 9, or 10 residues, more preferably at only 1, 2, or 3 residues, even more preferably at only 1 or 2 residues, yet even more preferably at only 1 residue.

8. The SARS-CoV-2 binding agent of any one of aspects 1-7, wherein the binding agent is an antibody, an antibody conjugate, or an antigen-binding fragment thereof.

9. The SARS-CoV-2 binding agent of any one of aspects 1-8, wherein the immunoglobulin heavy chain variable region polypeptide or single domain variable region polypeptide is humanized.

10. The SARS-CoV-2 binding agent of any one of aspects 1-9, wherein the binding agent comprises an antibody Fc region, preferably the Fc region of human IgG1.

11. The SARS-CoV-2 binding agent of any one of aspects 1-10, wherein the binding agent comprises at least two single domain antibodies or fragments thereof.

12. The SARS-CoV-2 binding agent of any one of aspects 1-11, wherein the binding agent is, or is part of, a multispecific or bispecific antibody or multivalent antibody.

13. The SARS-CoV-2 binding agent, wherein the binding agent binds to an epitope, wherein the epitope comprises amino acid residues 47, 50, 77, 80, 103, 173, and 178 relative to SEQ ID NO: 21.

14. The SARS-CoV-2 binding agent of any one of aspects 1-13, wherein the binding agent inhibits binding of SARS-CoV-2 to an ACE2 receptor.

15. A nucleic acid encoding the amino acid sequence of the SARS-CoV-2 binding agent of any one of aspects 1-14.

16. An isolated cell comprising the nucleic acid of aspect 15.

17. A method of providing a SARS-CoV-2 binding agent of any one of aspects 1-14, the method comprising expressing in a cell in vitro one or more nucleic acids encoding the SARS-CoV-2 binding agent.

18. A method of providing a SARS-CoV-2 binding agent of any one of aspects 1-14, the method comprising expressing in a cell in vivo one or more nucleic acids encoding the SARS-CoV-2 binding agent.

19. A composition comprising the SARS-CoV-2 binding agent of any one of aspects 1-14, and a pharmaceutically acceptable carrier.

20. A method for enhancing an immune response in a mammal, the method comprising administering to the mammal the SARS-CoV-2 binding agent of any one of aspects 1-14.

21. A method for treating a disease, disorder, or condition in a mammal that is responsive to inhibition of SARS-CoV-2 binding to an ACE2 receptor, the method comprising administering to the mammal the SARS-CoV-2 binding agent of any one of aspects 1-14.

22. A method for preventing a disease, disorder, or condition in a mammal that is responsive to inhibition of SARS-CoV-2 binding to an ACE2 receptor, the method comprising administering to the mammal the SARS-CoV-2 binding agent of any one of aspects 1-14.

23. The method of any one of aspects 20-22, wherein the route of administration is parenteral, oral, intravenous, intraperitoneal, subcutaneous, pulmonary, transdermal, intramuscular, intranasal, buccal, sublingual, or suppository, preferably intravenous or intranasal.

24. Use of a SARS-CoV-2 binding agent of any one of aspects 1-14 to prevent or treat a disease, disorder, or condition in a mammal that is responsive to inhibition of SARS-CoV-2 binding to an ACE2 receptor.

25. The method of any one of aspects 21-23 or the use of aspect 24, wherein the disease, disorder, or condition is COVID-19.

26. An isolated cell, hybridoma or cell line that expresses a SARS-CoV-2 binding agent of any one of aspects 1-14.

27. The isolated cell, hybridoma or cell line of aspect 26, wherein the isolated cell is suitable for administration to a subject, preferably a mammalian subject, more preferably a human subject.

The following example further illustrates the invention but, of course, should not be construed as in any way limiting its scope.

### Example

Camelid Monoclonal Antibody Production: Boost SARS-CoV2 BA.X lineage antigens were used to immunize llamas multiple times over a 3 month period. ELISA assays were performed to determine immune responses to antigens. Blood was removed from each animal (approximately 100 ml) and white cells were isolated by Ficoll gradient centrifugation. White cells were then used for RNA isolation (TRI-REAGENT SIGMA T-9424). cDNA was generated from RNA using specific primers for antibody amplification. Subsequent rounds of PCR were used to selectively amplify single domain antibody fragments which were then cloned into M13 phage display vectors. Vectors containing VHH-pIII protein fusions were transformed into E coli then treated with helper phage to reconstitute functional phage containing VHH fused to the viral coat wherein each phage contained the genetic instructions for expressing their specific VHH fragment.

M13 virus expression: VHH fragments were then used to pan ELISA plates coated with BA.X antigen. Non-binding phage were washed off the plates, while binding phage were eluted and added to E coli for infection and propagation. Additional rounds of panning and enrichment were used to concentrate binding phage. At each round of panning and enrichment, clones were sequenced to determine the specific VHH DNA sequence. Many of these sequences were then codon optimized for CHO cell expression and then synthesized in a human IgG1 Fc-containing vector wherein the VHH fragment is N-terminal to the Fc domain. These vectors were then transiently transfected into expiCHO cells using Mirus transfection reagents (MIR 6270, Mirus Madison WI). VHH-Fc protein was secreted and harvested from the media by protein A capture resin (Pro-A Resin, Lytic Solutions). Elutions were carried out at either pH 2.8 or 3.5 in glycine or acetate elution buffers respectively. Elution fractions were neutralized in Tris pH 7.2.

Analysis of VHH-Fc protein. Mesoscale (MSD) plates (MSD K15596U [Panel 26], K15617U [Panel 28], and K15679U [Panel 33]) and reader (MESO™ QuickPlex SQ 120) were used to analyze the VHH-Fc antibody interference with SARS-CoV2 RBD to ACE2 binding. The assay displays signal as ACE2 (labelled) binds to RBD (bound to the plate). Antibody that binds RBD interferes with ACE2 binding and lowers the signal in the assay. The identified VHH-Fc (SEQ ID NO: 3) demonstrates nearly complete inhibition of RBD-Ace2 binding at 5 ug/ml concentration as shown below, especially of late-arising variants such as BA.2, BA.4 and BA.5, XBB.1 and BQ.1.1. See Table 3. “BA.2+L452M” and “BA.2+L452R” are polypeptides with amino acid sequences identical to the the amino acid sequence of BA.2 except for the indicated substitution at the noted residue. **MSD Panel 33 samples; 9 test spots per well.

TABLE 3

| | | Concentration (ug/ml) | | | | | | | | |
|---|---|---|---|---|---|---|---|---|---|---|
| | | 5.000 | 1.677 | 0.550 | 0.183 | 0.061 | 0.020 | 0.007 | 0.002 | 0.001 |
| | | Concentration (nM) | | | | | | | | |
| | | 61.8 | 20.6 | 6.9 | 2.3 | 0.76 | 0.25 | 0.09 | 0.03 | 0.01 |
| Percent | BQ.1.1** | 99.9 | 99.9 | 99.9 | 99.9 | 97.9 | 51.0 | 20.2 | 15.7 | 12.3 |
| Inhibition of | XBB.1** | 99.9 | 99.9 | 99.9 | 99.9 | 99.1 | 60.6 | 21.5 | 17.0 | 15.8 |
| ACE2-RBD | BQ.1** | 99.9 | 99.9 | 99.9 | 99.9 | 97.6 | 52.9 | 14.3 | 25.2 | 12.6 |
| Binding* | BA.4.6/BF.7** | 99.9 | 99.9 | 99.9 | 99.9 | 96.6 | 47.5 | 18.4 | 20.9 | 15.8 |
| | BA.4/5 | 99.9 | 99.8 | 99.8 | 99.8 | 99.5 | 73.1 | 33.4 | 17.5 | 16.2 |
| | BA.4/5** | 99.9 | 99.8 | 99.7 | 99.6 | 95.0 | 49.1 | 11.2 | 18.2 | 16.0 |
| | BA.3 | 98.5 | 95.5 | 83.6 | 64.3 | 35.8 | 14.2 | 4.1 | 0 | 0 |
| | BA.2.12.1 | 99.9 | 99.9 | 99.9 | 99.8 | 98.8 | 62.8 | 26.5 | 12 | 4.1 |
| | BA.2/2.1/2.2/2.3/2.5/2.6/ | 99.9 | 99.9 | 99.9 | 99.9 | 99.2 | 69.8 | 30.9 | 13.3 | 12.6 |
| | 2.7/2.8/2.10/2.10.1/2.12 | | | | | | | | | |
| | BA.2.75** | 99.9 | 99.9 | 99.8 | 99.0 | 85.7 | 42.4 | 30.0 | 16.6 | 14.8 |
| | BA.2.75.2** | 99.9 | 99.9 | 99.9 | 99.8 | 97.8 | 61.8 | 26.6 | 27.3 | 10.2 |
| | BA.2 + L452M | 99.9 | 99.9 | 99.9 | 99.8 | 98.3 | 63.1 | 26.8 | 5.3 | 2.5 |
| | BA.2 + L452R | 99.9 | 99.9 | 99.9 | 99.8 | 99.4 | 71.6 | 30.4 | 5.6 | 0 |
| | B.1.1.529/BA.1/BA.1.15** | 96.4 | 91.3 | 78.9 | 58.5 | 20.4 | 5.7 | 0 | 0 | 0 |
| | Delta (B.1.617.2 & | 95.1 | 85.8 | 45.5 | 12.8 | 0 | 0 | 0 | 0 | 0 |
| | AY.3/4/4.2/5/6/7/12/14) | | | | | | | | | |
| | Beta (B.1.351/B.1.351.1) | 98.1 | 94.6 | 81.2 | 48.3 | 13.7 | 13.3 | 0 | 0 | 0 |
| | Alpha (B.1.1.7) | 97.6 | 93.8 | 78.0 | 43.5 | 15.3 | 8.9 | 2.0 | 3.0 | 4.2 |
| | Wuhan (A(WT)) | 94.7 | 86.9 | 54.6 | 19.0 | 0 | 0 | 0 | 0 | 0 |

In cases where the most recent variant SARS-CoV2 RBD was not available on a validated MSD assay plates to determine RBD-ACE2 interaction inhibition, those variant RBD proteins were expressed using transient transfection of CHO cells and tested the purified proteins for their binding to the VHH-Fc using a Sartorius Octet RH16 SPR instrument. This approach allowed the determination of dissociation contants (K_{DS}) for VHH-Fc binding to variant RBDs. The determined values are presented in Table 4 (see below). These data were consistent with data obtained via the MSD RBD-ACE2 binding platform and indicate broad and very potent binding of the VHH-Fc to SARS-CoV-2 variant RBDs.

TABLE 4

| Variant RBD | K_{D} (picomolar) |
|---|---|
| BA.5 | 3.6 |
| BQ.1.1 | 3.5 |
| XBB | 2.6 |
| BA.4.7 (BF.7) | <1* |
| XBB.1.5 | 1.1 |
| CH.1.1 | <1* |

*Values were below detection limit of the Sartorius instrument

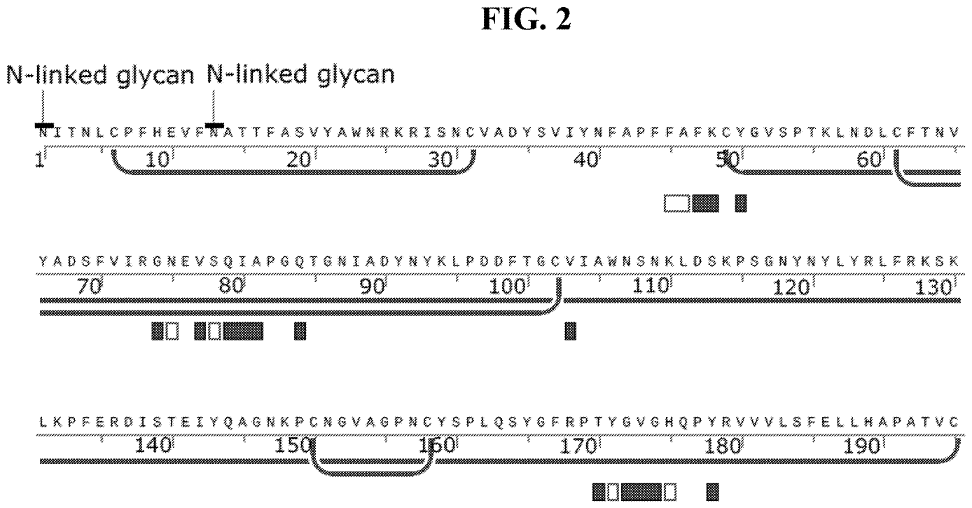

Computational methods were used to model the structure of the VHH-Fc/RBD interactions and provide insight into the RBD epitope for the VHH-Fc. AlphaFold-Multimer from Deepmind (Evans, R., et al., (2021) Protein complex prediction with AlphaFold-Multimer. bioRxiv 2021.10.04.463034; doi: doi.org/10.1101/2021.10.04.463034) was used. The RBD sequence from XBB.1.5 was used as an exemplary RBD. The resulting, best-fit model complex (confidence score of 0.56) suggested the VHH binds a depression of the RBD's molecular surface with possible contact with 21 residues of the RBD. Those SPIKE RBD contact residues include amino acid residues at positions 45-48, 50, 74, 75, 77-81, 84, 103, 170-175 and 178 relative to SEQ ID NO: 21. A schematic of the positioning of putative contact site residues relative to the primary amino acid sequence is given in FIG. 2. To be clear, residues 1-207 shown in FIG. 2 correspond to residues 1-207 of the RBD sequence of SEQ ID NO: 21. The figure aids to illustrate that the VHH epitope is comprised of residues from three distinct peptide regions of the RBD.

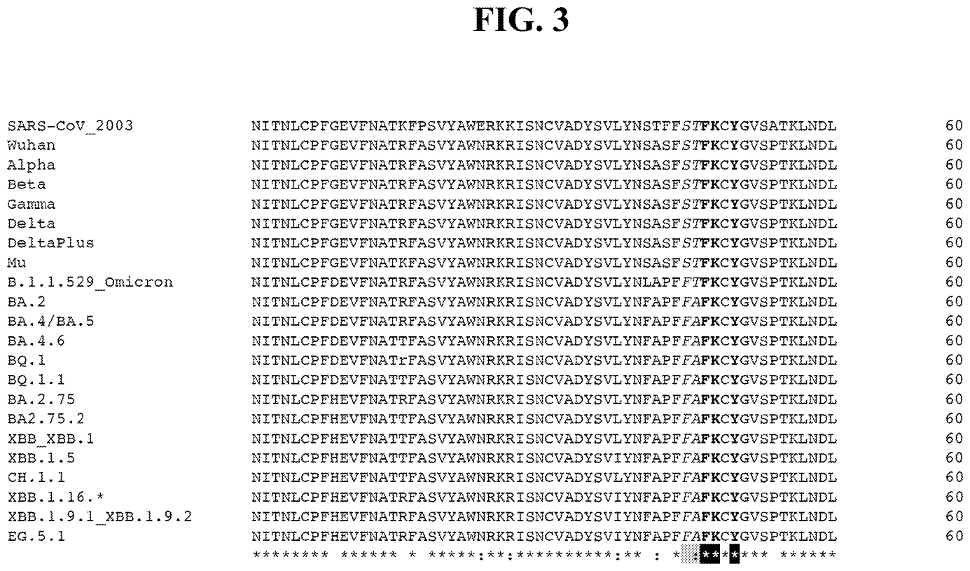

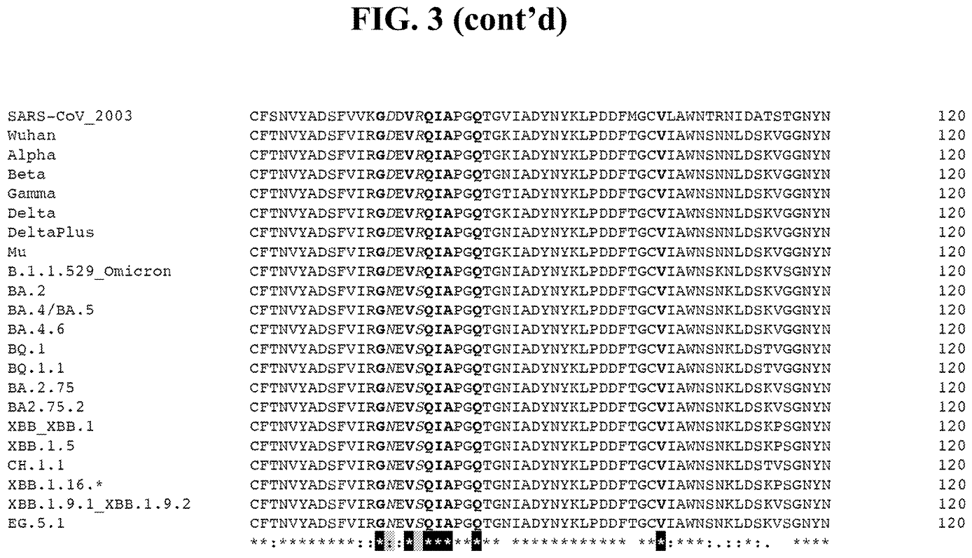

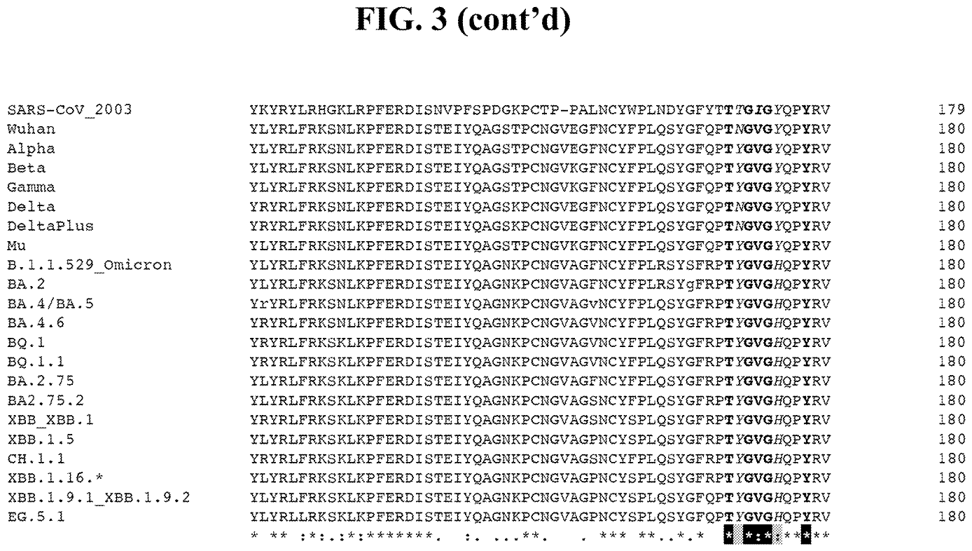

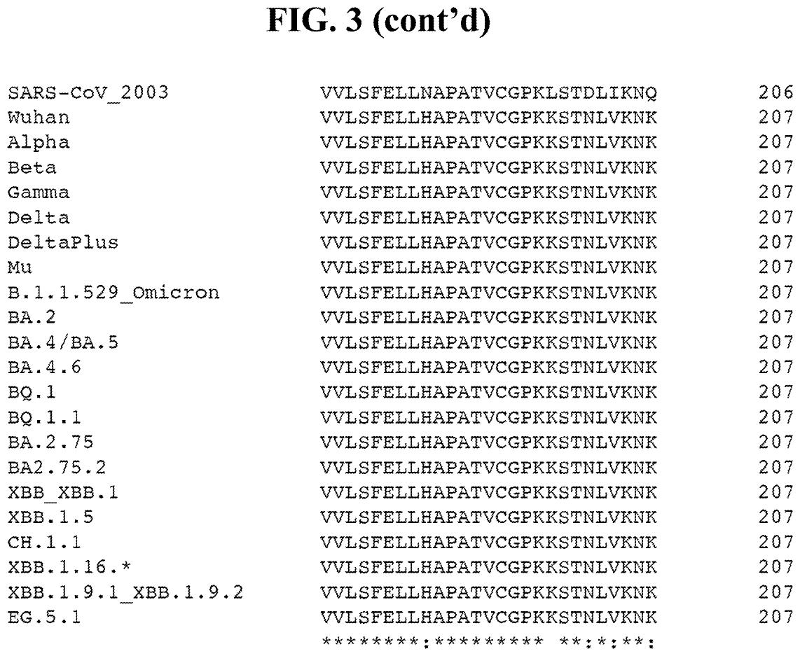

Alignments generated using CLUSTAL Omega of SARS-CoV RBD sequences were used to determine conservation of proposed epitope amino acids across major variants of SARS-CoV-2 and including SARS-CoV (2003). See FIG. 3. To be clear, residues 1-207 (1-206 with respect to SARS-CoV (2003)) shown in FIG. 3 correspond to residues 1-207 of the RBD sequence of SEQ ID NO: 21. This analysis shows a very high degree of conservation across variants for of the VHH epitope and this includes the SARS-CoV (2003).

The binding of the VHH to the original SARS-CoV (2003) RBD was verified through expressiong of the SARS-CoV (2003) RBD protein using transient transfection of CHO cells and testing the purified protein for its binding to the VHH-Fc using a Sartorius Octet RH16 SPR instrument. The K_{D} for interaction was determined to be 13 nM.

To determine the contribution of the proposed sites of homology in the binding interaction of VHH with the RBD, computational alanine scanning was performed. The analysis computationally determined the effect of changing one amino acid at a time to alanine and then remodeling and calulating its effect on the theoretical free-energy of binding. The results pointed to 9 RBD residues (Table 5) being key in VHH binding to it. Of which 7 are absolutely conserved across all known SARS-CoV-2 variants and 6 are conserved with the RBD from SARS-CoV (2003). Without wishing to be bound to theory, the two non-conserved sites likely are not involved in binding to a significant level because empirical VHH-RBD binding data and differences between RBD variants does not correlate with these amino acids changes. The VHH epitope is thus conserved across all known SARS-CoV-2 variant RBDs.

TABLE 5

| Variant | delta E | Prediction | Accessible | Contact | Conserved |
|---|---|---|---|---|---|
| F45A | 2.65 | destabilizing | surface | sidechain | no |
| F47A | 3.88 | destabilizing | surface | backbone | yes |
| Y50A | 2.48 | destabilizing | boundary | sidechain | yes |
| V77A | 2.03 | destabilizing | core | sidechain | yes |
| I80A | 3.21 | destabilizing | core | backbone | yes |
| V103A | 2.53 | destabilizing | core | sidechain | yes |
| Y171A | 1.71 | destabilizing | boundary | sidechain | no |
| V173A | 0.92 | destabilizing | boundary | backbone | yes |
| | | | | | (SARS-CoV-2; |
| | | | | | mutate V > |
| | | | | | I SARS- |
| | | | | | CoV 2003) |
| Y178A | 2.73 | destabilizing | core | sidechain | yes |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A SARS-CoV-2 binding agent comprising an immunoglobulin heavy chain variable region or single domain variable region comprising at least a complementarity determining region 3 (HCDR3) comprising any one of SEQ ID NOs 17 and 18.

2. (canceled)

3. A SARS-CoV-2 binding agent comprising an immunoglobulin heavy chain variable region or single domain variable region of any one of SEQ ID NOs: 1 and 2, wherein the immunoglobulin heavy chain variable region or single domain variable region comprises CDR sequences.

4. (canceled)

5. The SARS-CoV-2 binding agent of claim 1, wherein:
the single domain variable region polypeptide or the immunoglobulin heavy chain variable region polypeptide comprises a complementarity determining region 1 (CDR1) comprising SEQ ID NO: 5, a complementarity determining region 2 (CDR2) comprising SEQ ID NO: 12, and a complementarity determining region 3 (CDR3) comprising SEQ ID NO: 17, as determined via Chothia;
the single domain variable region polypeptide or the immunoglobulin heavy chain variable region polypeptide comprises a complementarity determining region 1 (CDR1) comprising SEQ ID NO: 6, a complementarity determining region 2 (CDR2) comprising SEQ ID NO: 11, and a complementarity determining region 3 (CDR3) comprising SEQ ID NO: 17, as determined via Kabat; or
the single domain variable region polypeptide or the immunoglobulin heavy chain variable region polypeptide comprises a complementarity determining region 1 (CDR1) comprising SEQ ID NO: 7, a complementarity determining region 2 (CDR2) comprising SEQ ID NO: 13, and a complementarity determining region 3 (CDR3) comprising SEQ ID NO: 18, as determined via IGMT.

6. The SARS-CoV-2 binding agent of claim 1 comprising an immunoglobulin heavy chain variable region polypeptide or a single domain variable region polypeptide that comprises an amino acid sequence that is at least 80% identical to any one of SEQ ID NOs: 1-4.

7. The SARS-CoV-2 binding agent of claim 1 comprising an immunoglobulin heavy chain variable region polypeptide or a single domain variable region polypeptide that comprises an amino acid sequence identical to any one of SEQ ID NOs: 1-4, except that the amino acid sequence differs at 1, 2, 3, 4, 5, 6, 7, 8, 9, 10, 11, 12, 13, 14, 15, 16, 17, 18, 19, or 20 residues.

8. The SARS-CoV-2 binding agent of claim 1, wherein the binding agent is an antibody, an antibody conjugate, or an antigen-binding fragment thereof.

9. The SARS-CoV-2 binding agent of claim 1, wherein the immunoglobulin heavy chain variable region polypeptide or single domain variable region polypeptide is humanized.

10. The SARS-CoV-2 binding agent of claim 1, wherein the binding agent comprises an antibody Fc region.

11. The SARS-CoV-2 binding agent of claim 1, wherein the binding agent comprises at least two single domain antibodies or fragments thereof.

12. The SARS-CoV-2 binding agent of claim 1, wherein the binding agent is, or is part of, a multispecific or bispecific antibody or multivalent antibody.

13. The SARS-CoV-2 binding agent of claim 1, wherein the binding agent inhibits binding of SARS-CoV-2 to an ACE2 receptor.

14. The SARS-CoV-2 binding agent, wherein the binding agent binds to an epitope, wherein the epitope comprises amino acid residues 47, 50, 77, 80, 103, 173, and 178 relative to SEQ ID NO: 21.

15. (canceled)

16. (canceled)

17. A method of providing a SARS-CoV-2 binding agent of claim 1, the method comprising expressing in a cell in vitro or in vivo one or more nucleic acids encoding the SARS-CoV-2 binding agent.

18. (canceled)

19. A composition comprising the SARS-CoV-2 binding agent of claim 1, and a pharmaceutically acceptable carrier.

20. A method for enhancing an immune response in a mammal, the method comprising administering to the mammal the SARS-CoV-2 binding agent of claim 1.

21. A method for preventing and/or treating a disease, disorder, or condition in a mammal that is responsive to inhibition of SARS-CoV-2 binding to an ACE2 receptor, the method comprising administering to the mammal the SARS-CoV-2 binding agent of claim 1.

22. (canceled)

23. The method of claim 21, wherein the route of administration is parenteral, oral, intravenous, intraperitoneal, subcutaneous, pulmonary, transdermal, intramuscular, intranasal, buccal, sublingual, or suppository, preferably intravenous or intranasal.

24. (canceled)

25. The method of claim 21, wherein the disease, disorder, or condition is COVID-19.

26. An isolated cell, hybridoma or cell line that expresses a SARS-CoV-2 binding agent of claim 1.

27. The isolated cell, hybridoma or cell line of claim 26, wherein the isolated cell is suitable for administration to a subject.
